SEQUENCE LISTING (1 sequence)

SEQ ID NO: 1 - protein, 4 aa, Synthetic construct
  source 1..4: note = amino acid spacer
GGFG

(19) United States
(12) Patent Application Publication
(10) Pub. No.: US 2026/0022127 A1
(43) Pub. Date: Jan. 22, 2026

(54) HEXACYCLIC TOPOISOMERASE INHIBITORS HAVING CYTOTOXIC ACTIVITY ON CANCER CELLS

(71) Applicant: NJ Bio, Inc., Princeton, NJ (US)

(72) Inventors: Nareshkumar Jain, Princeton, NJ (US); Allan Prior, Princeton, NJ (US); Steven L. Murkli, Princeton, NJ (US); Nabin Panth, Princeton, NJ (US); Arti Bhujade, Princeton, NJ (US)

(21) Appl. No.: 18/994,984

(22) PCT Filed: Jul. 21, 2023
(86) PCT No.: PCT/US2023/070686

§ 371 Date: Jan. 15, 2025

Related U.S. Application Data
(60) Provisional application No. 63/391,390, filed on Jul. 22, 2022.

(51) Int. Cl. C07D 491/22; A61K 31/4745; A61K 47/68; A61P 35/00

(52) U.S. Cl. CPC C07D 491/22; A61K 31/4745; A61K 47/68037; A61K 47/6849; A61K 47/6855; A61P 35/00

(57) ABSTRACT

Disclosed are topoisomerase inhibitor compounds as cytotoxic agents and further as pay loads for antibody-drug conjugates. Also disclosed are methods of making and use of the compounds in the treatment of proliferative diseases.

### CROSS-REFERENCE TO RELATED APPLICATIONS

This application is a U.S. National Stage application of PCT/US2023/070686, filed July 21, which claims the benefit of priority of, U.S. Provisional Patent Application No. 63/391,390, filed Jul. 22, 2022, the contents of which are incorporated herein by reference in their entirety for all purposes.

### SEQUENCE LISTING

The Instant Application contains a Sequence Listing which has been submitted electronically in XML format and is hereby incorporated by reference in its entirety. Said XML copy, created on Jan. 15, 2025, is named “NJB0005US2” and is 2,168 bytes in size. The Sequence Listing does not go beyond the disclosure in the application as filed.

### BACKGROUND

DNA topoisomerases are essential proteins that control DNA topology. Type I topoisomerases catalyze the formation and re-ligation of single-stranded (ss) breaks and Type II catalyze the formation and re-ligation of double-stranded (ds) DNA breaks. Human DNA topoisomerase I remains an important drug target for the treatment of cell proliferation diseases. Camptothecin is a well-known inhibitor of DNA topoisomerase I but suffers from solubility and stability issues. Derivatives and analogs of camptothecin, including irinotecan, topotecan, exatecan and its derivative deruxtecan, are also potent topoisomerase I inhibitors.

Antibody drug conjugates (ADC) include monoclonal antibodies (mAbs) attached to biologically active drugs using chemical linkers with labile bonds. The combination of cytotoxic active agents with the targeting capability of mAbs allows for the specific targeting of cancer cells.

Thus, there remains a need for novel, potent topoisomerase inhibitors and payloads for the development of ADCs for the potential use in the treatment of proliferative diseases, including use in cancer therapy.

### SUMMARY

Disclosed, in various non-limiting embodiments are topoisomerase inhibitors, methods of making, use of the inhibitors as payloads for ADCs, preparation of the ADCs, intermediate compounds, and ADCs formulated into pharmaceutical formulations; methods of use of the topoisomerase inhibitors, intermediate compounds, and ADCs for treatment of proliferative disorders, particularly in cancer therapy.

A compound of Formula (1), Formula (1′) or a pharmaceutically acceptable salt thereof,

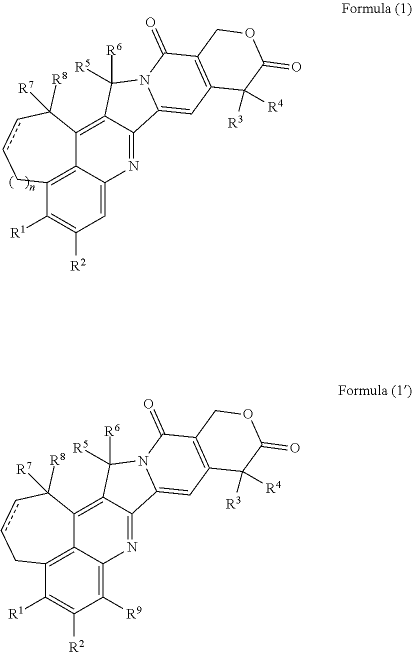

wherein
   R¹ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R¹ is C₁₋₆ alkyl, and more specifically R¹ is C₁ alkyl;
   R² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R² is halogen, and more specifically R² is F;
   R³ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R³ is hydroxyl, C₁₋₃ alkyl, or C₁ haloalkyl, and more specifically R³ is hydroxyl;
   R⁴ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R⁴ is C₁₋₃ alkyl or C₁₋₃ haloalkyl, and more specifically R⁴ is C₂ alkyl;
   wherein at least one of R³ and R⁴ is not H;
   R⁵ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁵ is H, hydroxyl, C₁₋₃ alkyl, C₂₋₃ alkenyl, C₂₋₃ alkynyl, (C₂₋₃ alkenyl)O—, or (C₂₋₃ alkynyl)O—, more specifically R⁵ is H, hydroxyl, or C₁ alkyl;
   R⁶ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁶ is H, hydroxyl, C₁₋₃ alkyl, C₂₋₃ alkenyl, C₂₋₃ alkynyl, —O(C₂₋₃ alkenyl), or —O(C₂₋₃ alkynyl), more specifically R⁶ is H, hydroxyl, or C₁ alkyl;
   or R⁵ and R⁶ together form an optionally substituted 5-6 member cyclic structure that is saturated or unsaturated, specifically a C₅₋₆ cycloalkyl or a C₅₋₆ cycloalkenyl;
   R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, —O—(C₁₋₃ alkyl)-NH—(C═O)—(C₁₋₃ alkyl)-NH₂, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁷ is H, hydroxyl, amino, C₁₋₃ alkyl, or halogen, more specifically R⁷ is H, hydroxyl, amino, C₁ alkyl, or chloro;
   R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, —O—(C₁₋₃ alkyl)-NH—(C═O)—(C₁₋₃ alkyl)-NH₂, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁸ is H, hydroxyl, amino, C₁₋₃ alkyl, or halogen, more specifically R⁸ is H, hydroxyl, amino, C₁ alkyl, or chloro;
   or R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
   R⁹ is H, C₁₋₆ alkyl, or halogen, specifically R⁹ is Cl; [symbol] is a single or double bond; and
   n is 0 or 1;
   with the following proviso:
   when R¹ is methyl, R² is fluoro, one of R³ and R⁴ is hydroxyl and the other is ethyl, R⁵ is H, R⁶ is H, [symbol] is a single bond, and n is 0, then when one of R⁷ and R⁸ is H, the other is not H, amino, mono-C₁₋₆ alkylamine, di-C₁₋₆ alkylamine, hydroxyl, or HO—(C₁₋₃ alkyl)-O—.

In another embodiment, a compound of

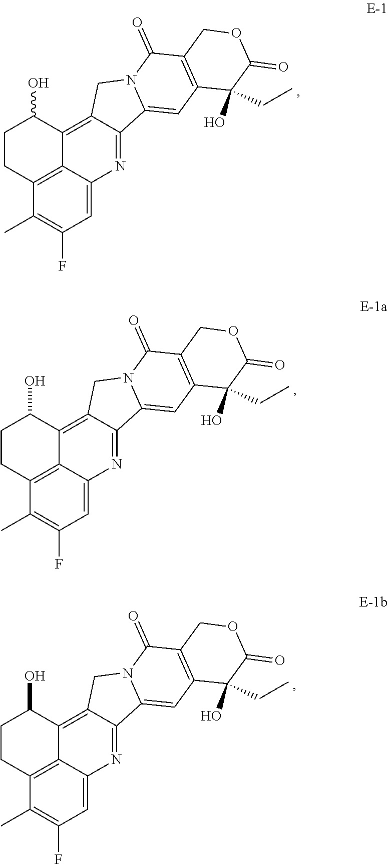

or
a pharmaceutically acceptable salt thereof.

In another embodiment, compound of Formula (A) or Formula (B)

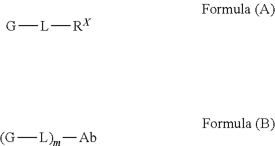

wherein
   L is a linking group;
   G is a structure of Formula (1) or Formula (1′), or a compound in Table 1, covalently attached to L through one of R¹, R², R³, R⁴, R⁵, R⁶, R⁷, or R⁸, specifically attached through R⁷ or R⁸;
   R^{X} is a reactive group suitable for forming a covalent bond to an antibody or antibody fragment;
   Ab is an antibody or antibody fragment; and
m is 1, 2, 3, 4, 5, 6, 7, or 8.

In another embodiment, a pharmaceutical composition comprises the compound of Formula (1), Formula (1′), or a pharmaceutically acceptable salt thereof, a compound of Formula (A), or a compound of Formula (B), and a pharmaceutically acceptable excipient.

In yet another embodiment, a method of treating a proliferative disease including a cancer, comprises administering to a patient in need thereof a compound of Formula (1), Formula (1′), or a pharmaceutically acceptable salt thereof, a compound of Formula (A), a compound of Formula (B), or a pharmaceutical formulation thereof.

These and other features and characteristics are more particularly described below.

### DETAILED DESCRIPTION

Disclosed are topoisomerase inhibitors, derivatives, and antibody drug conjugates of the novel compounds. The compounds and antibody drug conjugates find use as antiproliferative agents specifically as anticancer agents, optionally in the form of cytotoxic payloads for ADCs.

A compound of Formula (1), Formula (1′), or a pharmaceutically acceptable salt thereof,

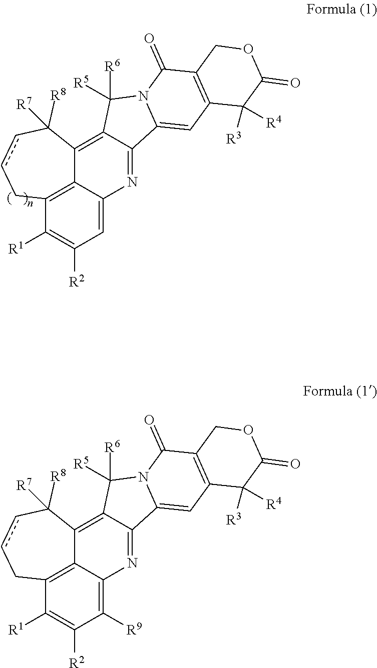

wherein
   R¹ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R¹ is C₁₋₆ alkyl, and more specifically R¹ is C₁ alkyl;
   R² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R² is halogen, and more specifically R² is F;
   R³ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R³ is hydroxyl, C₁₋₃ alkyl, or C₁ haloalkyl, and more specifically R³ is hydroxyl;
   R⁴ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R⁴ is C₁₋₃ alkyl or C₁₋₃ haloalkyl, and more specifically R⁴ is C₂ alkyl;
   R⁵ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁵ is H, hydroxyl, C₁₋₃ alkyl, C₂₋₃ alkenyl, C₂₋₃ alkynyl, (C₂₋₃ alkenyl)O—, or (C₂₋₃ alkynyl)O—, more specifically R⁵ is H, hydroxyl, or C₁ alkyl;
   R⁶ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁶ is H, hydroxyl, C₁₋₃ alkyl, C₂₋₃ alkenyl, C₂₋₃ alkynyl, —O(C₂₋₃ alkenyl), or —O(C₂₋₃ alkynyl), more specifically R⁶ is H, hydroxyl, or C₁ alkyl;
   or R⁵ and R⁶ together form an optionally substituted 5-6 member cyclic structure that is saturated or unsaturated, specifically a C₅₋₆ cycloalkyl or a C₅₋₆ cycloalkenyl;
   R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁷ is H, hydroxyl, amino, C₁₋₃ alkyl, or halogen, more specifically R⁷ is H, hydroxyl, amino, C₁ alkyl, or chloro;
   R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁸ is H, hydroxyl, amino, C₁₋₃ alkyl, or halogen, more specifically R⁸ is H, hydroxyl, amino, C₁ alkyl, or chloro;
   or R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
   R⁹ is H, C₁₋₆ alkyl, or halogen, specifically R⁹ is C₁;
   [symbol] is a single or double bond; and
   n is 0 or 1.

In an embodiment, the compound of Formula (1), Formula (1′), or a pharmaceutically acceptable salt thereof, as previously described, with the proviso that at least one of R³ and R⁴ is not H; or when R¹ is methyl, R² is fluoro, one of R³ and R⁴ is hydroxyl and the other is ethyl, R⁵ and R⁶ is H, n is 0, then neither R⁷ or R⁸ is H, amino, mono-C₁₋₆ alkylamine, or di-C₁₋₆ alkylamine.

In an embodiment, the compound of Formula (1), Formula (1′), or a pharmaceutically acceptable salt thereof, as previously described, with the proviso that neither R⁷ or R⁸ is amino, mono-C₁₋₆ alkylamine, or di-C₁₋₆ alkylamine.

In an embodiment, the compound of Formula (1), Formula (1′), or a pharmaceutically acceptable salt thereof, wherein [symbol] is a single bond, n is 0, R⁵ is H, R⁶ is H, and either R⁷ or R⁸ is —O—(C₁₋₃ alkyl)-NH—(C═O)—(C₁₋₃ alkyl)-NH₂ and the other is H, specifically wherein [symbol] is a single bond, n is 0, R⁵ is H, R⁶ is H, and either R⁷ or R⁸ is —O—(CH₂)—NH—(C═O)—(CH₂)—NH₂, and the other is H.

In an embodiment, the compound of Formula (1), or a pharmaceutically acceptable salt thereof, as previously described, with the proviso that when one of R⁷ and R⁸ is H, the other is not H, amino, mono-C₁₋₆ alkylamine, or di-C₁₋₆ alkylamine, hydroxyl, or HO—(C₁₋₃ alkyl)-O—.

In an embodiment, the compound of Formula (1), Formula (1′), or a pharmaceutically acceptable salt thereof, wherein R¹ is C₁₋₆ alkyl and specifically R¹ is C₁ alkyl. Further within this embodiment, n is 0 or n is 1.

In an embodiment, the compound of Formula (1), Formula (1′), or a pharmaceutically acceptable salt thereof, wherein R² is halogen and more specifically R² is F. Further within this embodiment, n is 0 or n is 1.

In an embodiment, the compound of Formula (1), Formula (1′), or a pharmaceutically acceptable salt thereof, wherein R¹ is C₁ alkyl and R² is F. Further within this embodiment, n is 0 or n is 1.

In an embodiment, the compound of Formula (1), Formula (1′), or a pharmaceutically acceptable salt thereof, wherein R³ is hydroxyl, C₁₋₃ alkyl, or C₁ haloalkyl, and more specifically R³ is hydroxyl. Further within this embodiment, n is 0 or n is 1.

In an embodiment, the compound of Formula (1), Formula (1′), or a pharmaceutically acceptable salt thereof, wherein R⁴ is C₁₋₃ alkyl or C₁₋₃ haloalkyl, and more specifically R⁴ is C₂ alkyl. Further within this embodiment, n is 0 or n is 1.

In an embodiment, the compound of Formula (1), Formula (1′), or a pharmaceutically acceptable salt thereof, wherein R³ is hydroxyl and R⁴ is C₂ alkyl. Further within this embodiment, n is 0 or n is 1.

In an embodiment, the compound of Formula (1), Formula (1′), or a pharmaceutically acceptable salt thereof, wherein R⁵ is H, hydroxyl, C₁₋₃ alkyl, C₂₋₃ alkenyl, C₂₋₃ alkynyl, (C₂₋₃ alkenyl)O—, or (C₂₋₃ alkynyl)O—, more specifically R⁵ is H, hydroxyl, or C₁ alkyl. Further within this embodiment, n is 0 or n is 1.

In an embodiment, the compound of Formula (1), Formula (1′), or a pharmaceutically acceptable salt thereof, wherein R⁶ is H, hydroxyl, C₁₋₃ alkyl, C₂₋₃ alkenyl, C₂₋₃ alkynyl, —O(C₂₋₃ alkenyl), or —O(C₂₋₃ alkynyl), more specifically R⁶ is H, hydroxyl, or C₁ alkyl. Further within this embodiment, n is 0 or n is 1.

In an embodiment, the compound of Formula (1), Formula (1′), or a pharmaceutically acceptable salt thereof, wherein R⁵ and R⁶ together form an optionally substituted 5-6 member cyclic structure that is saturated or unsaturated, specifically a C₅₋₆ cycloalkyl or a C₅₋₆ cycloalkenyl. Further within this embodiment, n is 0 or n is 1.

In an embodiment, the compound of Formula (1), Formula (1′), or a pharmaceutically acceptable salt thereof, wherein R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—. Further within this embodiment, n is O or n is 1.

In an embodiment, the compound of Formula (1), Formula (1′), or a pharmaceutically acceptable salt thereof, wherein R⁷ is H, hydroxyl, amino, C₁₋₃ alkyl, or halogen, and more specifically R⁷ is H, hydroxyl, C₁ alkyl, or chloro. Further within this embodiment, n is 0 or n is 1.

In an embodiment, the compound of Formula (1), Formula (1′), or a pharmaceutically acceptable salt thereof, wherein R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—. Further within this embodiment, n is 0 or n is 1.

In an embodiment, the compound of Formula (1), Formula (1′), or a pharmaceutically acceptable salt thereof, wherein R⁸ is H, hydroxyl, amino, C₁₋₃ alkyl, or halogen, more specifically R⁸ is H, hydroxyl, C₁ alkyl, or chloro. Further within this embodiment, n is 0 or n is 1.

In an embodiment, the compound of Formula (1), Formula (1′), or a pharmaceutically acceptable salt thereof, wherein R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic. Further within this embodiment, n is 0 or n is 1.

In an embodiment, the compound of Formula (1), Formula (1′), or a pharmaceutically acceptable salt thereof, wherein [symbol] is a single bond.

In an embodiment, the compound of Formula (1), Formula (1′), or a pharmaceutically acceptable salt thereof, wherein [symbol] is a double bond.

In an embodiment, the compound of Formula (1), Formula (1′), or a pharmaceutically acceptable salt thereof, wherein n is 0.

In an embodiment, the compound of Formula (1), Formula (1′), or a pharmaceutically acceptable salt thereof, wherein n is 1.

Exemplary compounds of Formula (1) and Formula (1′) are provided in Table 1. disclosed herein.

In an embodiment, a compound of Formula (A)

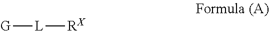

wherein
   L is a linking group;
   G is a structure of Formula (1) or Formula (1′) covalently attached to L through one of R¹, R², R³, R⁴, R⁵, R⁶, R⁷, or R⁸, specifically linked through R⁷ or R⁸; and
   R^{X} is a reactive group suitable for forming a covalent bond to an antibody or antibody fragment.

L, the linking group, can be a bond or a group containing 1 to about 250 non-hydrogen atoms including C, N, O, S, halogen, or a combination thereof; further wherein L can optionally include one or more groups including an ether, thioether, amide, carbonyl, ester, carbonate, carbamate, urea, or a combination thereof. In an embodiment, L comprises an ethylene glycol unit, specifically about 2 to about 25 repeating ethylene glycol units, more specifically about 5 to about 10 repeating ethylene glycol units; an amino acid unit, specifically 1 to about 12 amino acid units, more specifically about 2 to about 10 amino acid units, and yet more specifically about 2 to about 3 amino acid units; or a combination thereof.

Within the amino acid groups of L, each amino acid unit can be arginine, histidine, lysine, aspartic acid, glutamic acid, serine, threonine, asparagine, glutamine, cysteine, selenocystein, glycine, proline, alanine, valine, isoleucine, leucine, methionine, phenylalanine, tyrosine, tryptophan, or citrulline (Cit). The amino acid unit may be enzymatically cleaved by one or more enzymes, including a tumor-associated protease. In an embodiment, the amino acid unit is alanine-valine, valine-alanine, valine-citrulline, or citrulline-valine.

The R^{X} group is a reactive group capable of attaching a compound such as Formula (1) to an antibody or antibody fragment. The R^{X} may be an amine, —O—NH₂, maleimide, azide, 2,5-dioxopyrrolidin-1-yl formate,

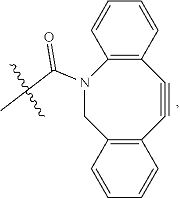 [SMILES: *C(=O)N1Cc2ccccc2C#Cc2ccccc21]

thiol, pentafluorophenyl ester, a carbonate, and the like. Suitable carbonate reactive groups include 4-nitrophenylcarbonate, 1-[6-trifluoromethylbenzotriazolyl]carbonate, 2-pyridylcarbonate, N-succinimidyl carbonate, and the like.

In an embodiment, the compound of Formula (A), as previously described, with the proviso that G is not a compound of Formula (1) where R⁷ or R⁸ is H and the other is hydroxyl or HO—(C₁₋₃ alkyl)-O—.

In an embodiment, a compound of Formula (B)

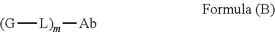

wherein
   L is a linking group as previously described;
   G is a structure of Formula (1) or Formula (1′) covalently attached to L through one of R¹, R², R³, R⁴, R⁵, R⁶, R⁷, or R⁸, specifically linked through R⁷ or R⁸;
   Ab is an antibody or antibody fragment; and
   m is 1, 2, 3, 4, 5, 6, 7, or 8.

Ab is an antibody or antibody fragment that preferentially binds to a target cell. “Antibody” as herein includes monoclonal antibodies, polyclonal antibodies, dimers, multimers, multispecific antibodies (e.g., bispecific antibodies), and antibody fragments that exhibit the desired biological activity. Antibodies can be murine, human, humanized, chimeric, or derived from other species. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

In an embodiment, the antibody-drug conjugate of Formula (B) has a drug loading of drug, e.g. compound of Formula (1) or Formula (1′), to antibody/antibody fragment (Ab) of from 1 to about 8, specifically about 2 to about 6, more specifically about 3 to about 4.

In an embodiment, the compound of Formula (B), as previously described, with the proviso that G is not a compound of Formula (1) where R⁷ or R⁸ is H and the other is hydroxyl or HO—(C₁₋₃ alkyl)-O—.

Exemplary compounds of Formula (A) are provided in Table 2. disclosed herein.

Disclosed herein are pharmaceutical formulations comprising a compound of Formula (1), compound of Formula (A), or compound of Formula (B), specifically an antibody-drug conjugate of Formula (B).

The compound of Formula (1), compound of Formula (1′), compound of Formula (A), or Formula (B), are cytotoxic compounds suitable for use to treat proliferative diseases, specifically as anticancer agents.

The compounds are described using standard nomenclature. Unless defined otherwise, all technical and scientific terms used herein have the same meaning as is commonly understood by one of ordinary skill in the art to which this disclosure belongs. Unless clearly contraindicated by the context each compound name includes the free acid or free base form of the compound as well hydrates of the compound and all pharmaceutically acceptable salts of the compound.

The terms “Formula (1),” “Formula (1′),” “Formula (A),” and “Formula (B)”, etc., as used herein, encompass all compounds that satisfy Formula (1), Formula (1′) Formula (A), and Formula (B), including any enantiomers, racemates and stereoisomers, as well as all pharmaceutically acceptable salts and radioisotopes of such compounds. The phrases “a compound of Formula (1),” “a compound of Formula (1′),” “a compound of Formula (A),” and “a compound of Formula (B)” include all subgeneric groups of Formula (1), Formula (1′), Formula (A), and Formula (B), and so forth, as well as all forms of such compounds, including salts and hydrates, unless clearly contraindicated by the context in which this phrase is used.

Formula (1) and Formula (1′) include all subformulae thereof. In certain situations, the compounds of Formula (1) or Formula (1′) may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In these situations, single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example, a chiral high performance liquid chromatography (HPLC) column.

Where a compound exists in various tautomeric forms, the compound is not limited to any one of the specific tautomers, but rather includes all tautomeric forms.

All isotopes of atoms occurring in the present compounds are contemplated. Isotopes include those atoms having the same atomic number but different mass numbers. By way of general example, and without limitation, isotopes of hydrogen include tritium and deuterium; isotopes of carbon include ¹¹C, ¹³C, and ¹⁴C; and an isotope of fluorine includes ¹⁸F.

A dash (“—”) that is not between two letters or symbols is used to indicate a point of attachment for a substituent. For example, —(CH₂)C₃-C₈cycloalkyl is attached through carbon of the methylene (CH₂) group.

“Alkanoyl” is an alkyl group as defined herein, covalently bound to the group it substitutes by a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

The term “alkyl”, as used herein, means a branched or straight chain saturated aliphatic hydrocarbon group having the specified number of carbon atoms, generally from 1 to about 12 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₆alkyl, C₁-C₄alkyl, and C₁-C₂alkyl. When C₀-Cₙ alkyl is used herein in conjunction with another group, for example, (cycloalkyl) C₀-C₄alkyl, the indicated group, in this case cycloalkyl, is either directly bound by a single covalent bond (Co), or attached by an alkyl chain having the specified number of carbon atoms, in this case 1, 2, 3, or 4 carbon atoms. Examples of alkyl include, but are not limited to, methyl, ethyl, n-propyl, isopropyl, n-butyl, 3-methylbutyl, t-butyl, n-pentyl, and sec-pentyl.

The term “alkoxy” represents an alkyl group as defined above with the indicated number of carbon atoms attached through an oxygen bridge. Examples of alkoxy include methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

The term “aryl”, as used herein, means aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Bicyclic aryl groups may be further substituted with carbon or non-carbon atoms or groups. Bicyclic aryl groups may contain two fused aromatic rings (naphthyl) or an aromatic ring fused to a 5- to 7-membered non-aromatic cyclic group that optionally contains 1 or 2 heteroatoms independently chosen from N, O, and S, for example, a 3,4-methylenedioxy-phenyl group. Aryl groups include, for example, phenyl, naphthyl, including 1-naphthyl and 2-naphthyl, and bi-phenyl.

The term “cycloalkyl”, as used herein, indicates a saturated hydrocarbon ring group, having only carbon ring atoms and having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane.

The term “cycloalkenyl”, as used herein, means a saturated hydrocarbon ring group, comprising one or more unsaturated carbon-carbon bonds, which may occur in any stable point of the ring, and having the specified number of carbon atoms. Monocyclic cycloalkenyl groups typically have from 3 to about 8 carbon ring atoms or from 3 to 7 (3, 4, 5, 6, or 7) carbon ring atoms. Cycloalkenyl substituents may be pendant from a substituted nitrogen or carbon atom, or a substituted carbon atom that may have two substituents may have a cycloalkenyl group, which is attached as a spiro group. Examples of cycloalkenyl groups include cyclopropenyl, cyclobutenyl, cyclopentenyl, or cyclohexenyl as well as bridged or caged saturated ring groups such as norbornene.

The term “heteroaryl”, as used herein, indicates a stable 5- to 7-membered monocyclic or 7- to 10-membered bicyclic heterocyclic ring which contains at least 1 aromatic ring that contains from 1 to 4, or specifically from 1 to 3, heteroatoms chosen from N, O, and S, with remaining ring atoms being carbon. When the total number of S and O atoms in the heteroaryl group exceeds 1, theses heteroatoms are not adjacent to one another. Specifically, the total number of S and O atoms in the heteroaryl group is not more than 2, more specifically the total number of S and O atoms in the heteroaryl group is not more than 1. A nitrogen atom in a heteroaryl group may optionally be quaternized. When indicated, such heteroaryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a [1,3]dioxolo[4,5-c]pyridyl group. In certain embodiments 5- to 6-membered heteroaryl groups are used. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, and 5,6,7,8-tetrahydroisoquinoline.

“Haloalkyl” includes both branched and straight-chain alkyl groups having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined herein attached through an oxygen bridge (oxygen of an alcohol radical).

“Halo” or “halogen” is any of fluoro, chloro, bromo, and iodo.

“Mono- and/or di-alkylamino” is a secondary or tertiary alkyl amino group, wherein the alkyl groups are independently chosen alkyl groups, as defined herein, having the indicated number of carbon atoms. The point of attachment of the alkylamino group is on the nitrogen. Examples of mono- and di-alkylamino groups include ethylamino, dimethylamino, and methyl-propyl-amino.

The term “substituted”, as used herein, means that any one or more hydrogens on the designated atom or group is replaced with a selection from the indicated group, provided that the designated atom's normal valence is not exceeded. When the substituent is oxo (i.e., ═O) then 2 hydrogens on the atom are replaced. When an oxo group substitutes aromatic moieties, the corresponding partially unsaturated ring replaces the aromatic ring. For example, a pyridyl group substituted by oxo is a pyridone. A stable compound or stable structure is meant to imply a compound that is sufficiently robust to survive isolation from a reaction mixture, and subsequent formulation into an effective therapeutic agent.

Unless otherwise specified, e.g. when a dash (“—”) is used, substituents are named into the core structure. For example, it is to be understood that when (cycloalkyl)alkyl is listed as a possible substituent the point of attachment of this substituent to the core structure is in the alkyl portion, or when arylalkyl is listed as a possible substituent the point attachment to the core structure is the alkyl portion.

Suitable groups that may be present on a “substituted” or “optionally substituted” position include, but are not limited to, halogen; cyano; hydroxyl; nitro; azido; alkanoyl (such as a C₂-C₆ alkanoyl group such as acyl or the like); carboxamido; alkyl groups (including cycloalkyl groups) having 1 to about 8 carbon atoms, or 1 to about 6 carbon atoms; alkenyl and alkynyl groups including groups having one or more unsaturated linkages and from 2 to about 8, or 2 to about 6 carbon atoms; alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being an exemplary arylalkyl group; arylalkoxy having 1 to 3 separate or fused rings and from 6 to about 18 ring carbon atoms, with benzyloxy being an exemplary arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothienyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

The term “pharmaceutically acceptable salt”, as used herein, includes derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The compounds of Formula (1) and its conjugates may be formulated with one or more excipients and prepared into pharmaceutical formulations for any suitable route of administration including oral or parenteral administration. As used herein, parenteral administration includes intravenous, cutaneous, subcutaneous, intramuscular, and the like.

The pharmaceutical composition generally comprises the compound of Formula (1), or its conjugates, and a pharmaceutically acceptable excipient, including carriers, buffers, antioxidants, and the like.

Pharmaceutical formulations for oral administration include tablets, capsules, powders, liquid, semisolids, and the like. Known pharmaceutically acceptable excipients used for oral administration may be used.

For parenteral administration, including intravenous or other injection, the formulation will comprise the compound of Formula (1), or its conjugate, and a parenterally acceptable aqueous solution. Parenteral solutions are to be pyrogen-free and have suitable pH, isotonicity, and stability. Suitable parenteral vehicles include Sodium Chloride Injection, Ringer's Injection, Lactated Ringer's Injection, and the like. The parenteral formulation may further comprise an antioxidant, a buffer, a preservative, a stabilizer, or a combination thereof.

The compounds and compositions disclosed herein find use for treating cancer, and in particular, in the form of ADCs for cancer therapy.

“Cancer” as used herein can refer to adenocarcinomas, carcinomas, leukemias, lymphomas, sarcomas, solid and lymphoid cancers, and the like. Examples of different types of cancer include, acute lymphocytic leukemia (acute lymphoblastic leukemia), acute myeloid leukemia (acute myelogenous leukemia, acute myeloblastic leukemia, acute myelocytic leukemia, acute granulocytic leukemia, and acute nonlymphocytic leukemia), anal cancer, B-cell lymphoma, bile duct cancer, bladder cancer, blood cancer, bone cancer, breast cancer, Burkitt's lymphoma, central nervous system cancer, cervical cancer, choriocarcinoma, chronic lymphocytic leukemia, chronic myeloid leukemia (chronic myelogenous leukemia), colon cancer, colorectal cancer, endometrial cancer, epithelial cancer, esophageal cancer, fibrosarcoma, gall bladder cancer, gastrointestinal carcinoid tumors, glioma, head and neck cancer, Large Cell lymphoma, liposarcoma, liver cancer (i.e., hepatocarcinoma), lung cancer (e.g., non-small cell lung cancer or NSCLC), melanoma, monocytic leukemia, multiple myeloma, myelodisplastic syndromes (MDS), myelogenous leukemia, neuroblastoma, non-Hodgkin's lymphoma, ovarian cancer, osteogenic sarcoma, pancreatic cancer, pleural cancer, prostate cancer, rectal cancer, renal cancer (i.e., renal cell carcinoma), skin cancer, Small Cell lymphoma, small intestine cancer, stomach (gastric) cancer, testicular cancer, thyroid cancer, uterine cancer, and the like.

The patient may be a mammalian patient, specifically a human.

The dosage amount of the compound of Formula (1) or Formula (1′) for the treatment of a disease, such as a proliferative disease, will depend on the disease to be treated, the severity of the disease, previous therapy and concurrent therapy, the patient's clinical history, and other factors. The Formula (1) or Formula (1′), or its conjugate, can be administered to the patient at one time or over a series of treatments over days, weeks, or months. In an exemplary amount, about 1 μg/kg to about 15 mg/kg of the compound of Formula (1) or Formula (1′) may be a starting dosage for administration to the patient. The dosage may be by one or more separate administrations, or by continuous infusion. An exemplary daily dosage might range from about 1 μg/kg to about 100 mg/kg or more, depending on the factors previously discussed. An exemplary dosage of the compound of Formula (1) or Formula (1′) to be administered to a patient can be in the range of about 0.1 to about 10 mg/kg of patient weight. In an embodiment, an exemplary dosing regimen comprises a course of administering an initial loading dose of about 4 mg/kg, followed by additional doses every week, two weeks, or three weeks of the compound of Formula (1) or Formula (1′).

In an embodiment, a pharmaceutical composition comprises the compound of Formula (1), Formula (1′), or a pharmaceutically acceptable salt thereof, or a compound of Formula (A), and a pharmaceutically acceptable excipient.

In yet another embodiment, a method of treating a proliferative disease including a cancer, comprises administering to a patient in need thereof a compound of Formula (1), Formula (1′), or a pharmaceutically acceptable salt thereof, a compound of Formula (A), or a compound of Formula (B), or a pharmaceutical formulation thereof.

The following examples are merely illustrative of the topoisomerase inhibitor compounds disclosed herein and are not intended to limit the scope hereof.

### EXAMPLES

General Solvents and reagents were purchased from Sigma-Aldrich, VWR, or Fisher Scientific, and used without further purification. Reactions were monitored either by thin-layer chromatography (TLC) or by analytical liquid chromatography-mass spectrometry (LC-MS) employing a Waters Acquity Ultra Performance LC system and a Synapt high-definition mass spectrometer. ¹H NMR spectra were recorded on an Oxford AS400 magnet connected to a Varian Mercury console (300 MHz). All chemical shifts are reported in ppm and coupling constants, J, are reported in hertz (Hz). NMR solvent peaks were referenced as follows: (¹H NMR) CDCl₃: 7.27 ppm, DMSO-d₆: 2.50 ppm. Compounds were purified by flash column chromatography on a Teledyne ISCO Combi-Flash system using normal phase silica gel (SiliCycle Inc.) or reverse phase (Teledyne Gold-C18 or C18Aq) pre-packed columns. The purity of compounds was determined by analytical HPLC (Waters Acquity Ultra Performance) using an Acquity UPLC CSH C18 1.7 μm (50 mm×2.1 mm) column and flow rate of 0.3 mL/min. Gradient conditions: solvent A (0.1% formic acid in water) and solvent B (0.1% formic acid in acetonitrile): 0-0.1 min 95% A, 0.1-4.0 min 5-95% B (linear gradient), 4.0-5.0 min 95% B, UV detection at 254 nm and 220 nm.

### Example 1

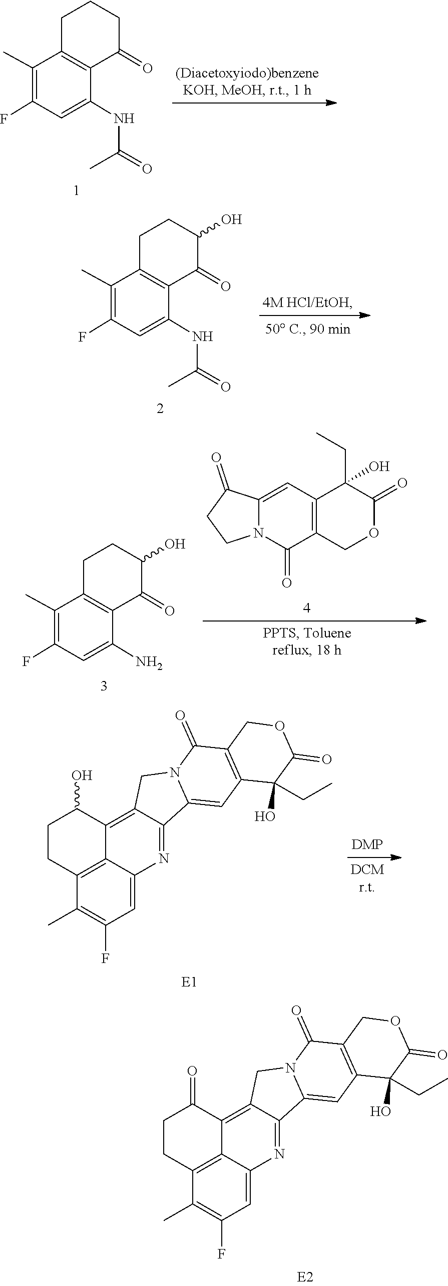

### Synthesis of Compound 2

Compound 1 (CAS #143655-58-7; 0.5 g, 2.13 mmol) was added to a solution of KOH (358 mg, 6.39 mmol) in MeOH (15 mL) at 0° C. and stirred until a solution formed. Diacetoxyiodobenzene (1.51 g, 4.68 mmol) was added in one portion at 0° C. and the reaction was allowed to warm to ambient temperature and stir for 1 h. The reaction was concentrated in vacuo and purified by reverse phase C18 flash chromatography (gradient elution; 15% acetonitrile (ACN)/water-100% ACN with 0.05% formic acid modifier) to afford compound 2 (410 mg, 1.63 mmol, 77%) as an off-white solid. MS (ESI) m/z: 252.0 [M+H]⁺.

### Synthesis of Compound 3

Compound 2 (70 mg, 0.279 mmol) was dissolved in 4M HCl in EtOH (5.0 mL) and stirred at 50° C. for 90 min. The reaction was cooled to ambient temperature, basified to pH 14 using 1M NaOH (aq.) and extracted into dichloromethane (DCM) (20 mL×3). The combined organic layers were washed with brine (×1), dried (Na₂SO₄), filtered and concentrated in vacuo to afford compound 3 (50 mg, 0.24 mmol, 86%) as a purple solid. MS (ESI) m/z: 210.0 [M+H]⁺.

### Synthesis of Compound E-1 (General Cyclization Procedure)

An oven dried reaction vessel equipped with reflux condenser was charged with compound 3 (500 mg, 2.39 mmol), compound 4 (CAS #110351-94-5; 629 mg, 2.39 mmol), pyridinium p-toluenesulfonate (PPTS) (60 mg, 0.24 mmol) and anhydrous toluene (12 mL). The mixture was refluxed for 24 h under atmosphere of nitrogen. The reaction was concentrated in vacuo, and the crude solid was purified by silica gel flash chromatography (gradient elution; 100% DCM-20% MeOH/DCM) to afford compound E-1 (250 mg, 0.57 mmol, 24%) as a yellow solid. MS (ESI) m/z: 437.0 [M+H]⁺.

### Synthesis of Compound E-2

Dess-Martin periodinane (DMP) (175 mg, 0.413 mmol) was added to a suspension of compound E-1 (150 mg, 0.344 mmol) in anhydrous DCM (25 mL). The reaction was stirred at ambient temperature for 1 h then purified on silica gel flash chromatography (gradient elution; 100% DCM-20% MeOH/DCM) to afford a mixture of compound E-2 and DMP by-product. The mixture was triturated with EtOAc to provide compound E-2 (100 mg, 0.23 mmol, 67%) as an orange solid. MS (ESI) m/z: 435.0 [M+H]⁺.

### Synthesis of E-18

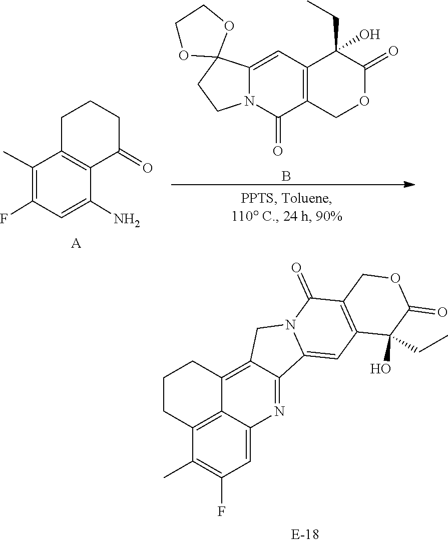

Aniline A (CAS #210346-39-7) was condensed with ketal B (CAS #110351-93-4) using the general cyclization conditions (See E-1 synthesis) to afford E-18 in 90% yield. MS (ESI) m/z: 421.0 [M+H]⁺.

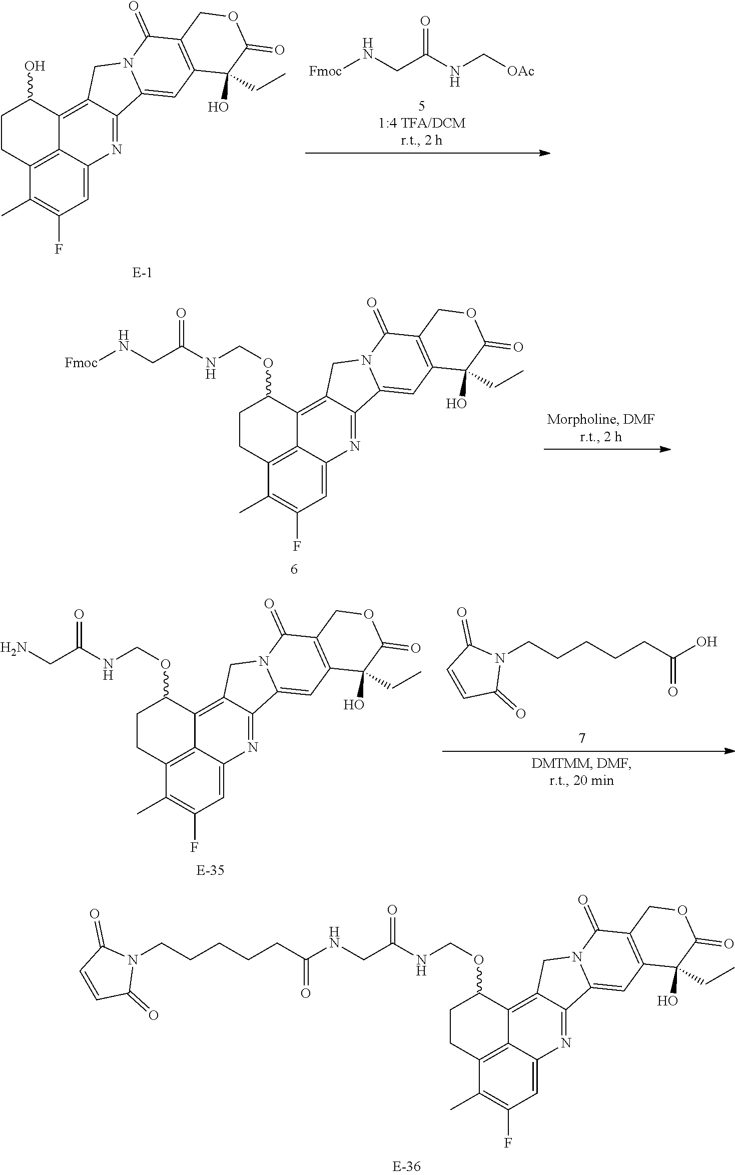

### Synthesis of Compound 6

Compounds E-1 (80 mg, 0.183 mmol) and 5 (CAS #1599440-06-8; 101 mg, 0.275 mmol) were dissolved in anhydrous DCM/trifluoroacetic acid (TFA) (4:1) (40 mL) and stirred at ambient temperature for 30 min under nitrogen protection. The solution was concentrated in vacuo to provide an oil that was purified by reverse phase C18 flash chromatography (gradient elution; 30% ACN/water-100% ACN) to afford compound 6 (40 mg, 0.054 mmol, 29%) as a light brown solid. MS (ESI) m/z: 745.0 [M+H]⁺.

### Synthesis of Compound E-35

Compound 6 (160 mg, 0.215 mmol) was dissolved in 1:4 morpholine/dimethylformamide (DMF) (2 mL) and the solution was stirred at ambient temperature for 2 h. Cold diethyl ether (20 mL) was added, and the mixture was placed in the freezer overnight. The solids were collected by centrifugation (6000 rpm, 10 min). The supernatant was decanted, and solids washed with cold diethyl ether (5 mL×1) then dried under high vacuum to afford E-35 (80 mg, 0.153 mmol, 71%) as a light brown solid. MS (ESI) m/z: 523.0 [M+H]⁺.

### Synthesis of Compound E-36

(4-(4,6-Dimethoxy-1,3,5-triazin-2-yl)-4-methyl-morpholinium chloride) (DMTMM) (15.9 mg, 0.0575 mmol) was added to a solution of E-35 (25 mg, 0.0479 mmol) and compound 7 (CAS #55750-53-3; 11.1 mg, 0.0527 mmol) in DMF. The reaction was stirred at ambient temperature for 20 min. Cold diethyl ether (30 mL) was added to precipitate a solid which collected by centrifugation (6000 rpm, 5 min) and solid was purified on silica gel flash chromatography (gradient elution; 100% DCM-25% MeOH/DCM) to afford compound E-36 (8.0 mg, 0.011 mmol, 23%) as a white solid. MS (ESI) m/z: 716.3 [M+H]⁺.

### Synthesis of Compound E-37

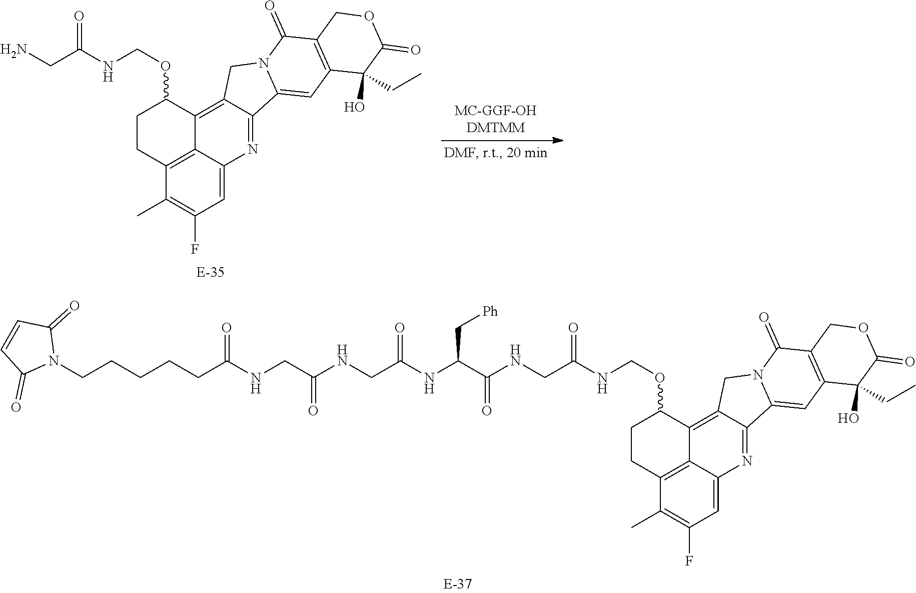 [SMILES: CC[C@@]1(O)C(=O)OCc2c1cc1n(c2=O)Cc2c-1nc1cc(F)c(C)c3c1c2C(OCNC(=O)CN)CC3 ; CC[C@@]1(O)C(=O)OCc2c1cc1n(c2=O)Cc2c-1nc1cc(F)c(C)c3c1c2C(OCNC(=O)CNC(=O)[C@H](Cc1ccccc1)NC(=O)CNC(=O)CNC(=O)CCCCCN1C(=O)C=CC1=O)CC3]

DMTMM (12.7 mg, 0.0460 mmol) was added to a solution of E-35 (20 mg, 0.0383 mmol) and MC-GGF-OH (CAS #1599440-15-9) (18.1 mg, 0.0383 mmol) in DMF and the reaction was stirred at ambient temperature for 20 min. Cold diethyl ether (40 mL) was added to precipitate a solid which was collected by centrifugation (6000 rpm, 5 min) and solid was purified by silica gel flash chromatography (gradient elution; 100% DCM-25% MeOH/DCM) to afford compound E-37 (18 mg, 0.018 mmol, 48%) as a white solid. MS (ESI) m/z: 977.4 [M+H]⁺.

### Separation of E-35a* and E-35b*

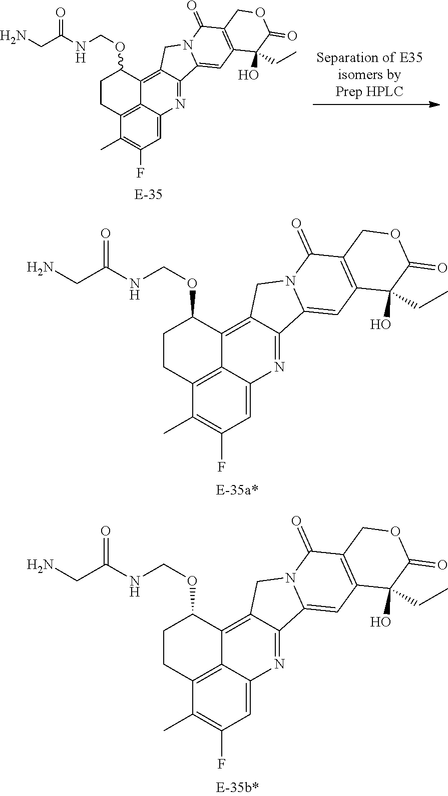 [SMILES: CC[C@@]1(O)C(=O)OCc2c1cc1n(c2=O)Cc2c-1nc1cc(F)c(C)c3c1c2C(OCNC(=O)CN)CC3 ; CC[C@@]1(O)C(=O)OCc2c1cc1n(c2=O)Cc2c-1nc1cc(F)c(C)c3c1c2[C@H](OCNC(=O)CN)CC3 ; CC[C@@]1(O)C(=O)OCc2c1cc1n(c2=O)Cc2c-1nc1cc(F)c(C)c3c1c2[C@@H](OCNC(=O)CN)CC3]

A stock solution of E35 was prepared by dissolving 200 mg of E35 in 2:1:1 dmso/acetonitrile/water (80 mL). The E35 isomers were separated using C18 prep-HPLC (Phenomenex C18 Luna column; 250 mm×50 mm; 30% acetonitrile in water-isocratic with 0.1% TFA; flow rate of 150 ml/min; 4 mL of the stock solution was used per injection; total of 20 injections). The combined peak 1 fractions were lyophilized to give E35-a* (peak 1) as a pale yellow solid (18 mg) MS (ESI) m/z: 523.3 [M+H]⁺. The combined peak 2 fractions were lyophilized to give E35-b* (peak 2) as a pale yellow solid (31 mg) MS (ESI) m/z: 523.1 [M+H]⁺. The absolute configuration of each isomer was not determined.

### Synthesis of E-39

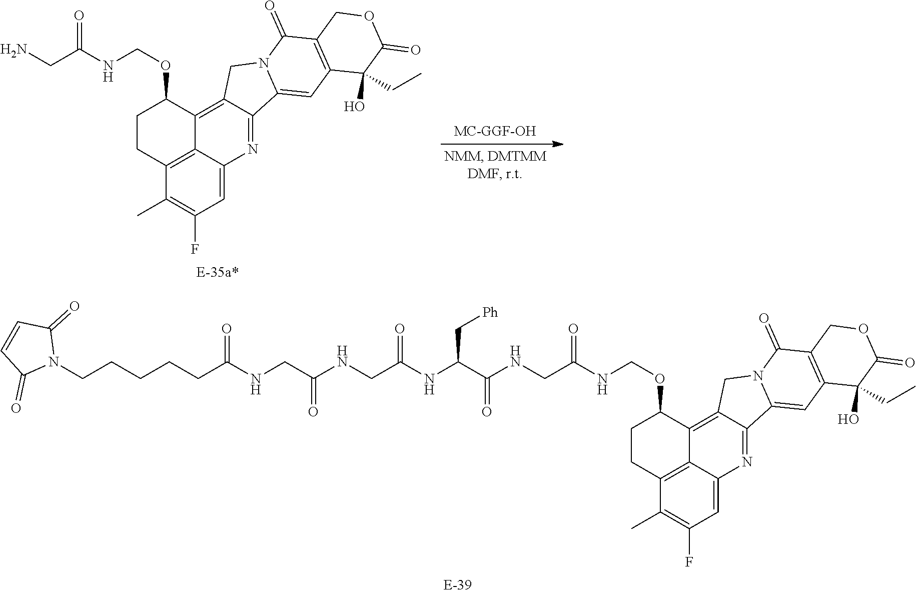 [SMILES: CC[C@@]1(O)C(=O)OCc2c1cc1n(c2=O)Cc2c-1nc1cc(F)c(C)c3c1c2[C@H](OCNC(=O)CN)CC3 ; CC[C@@]1(O)C(=O)OCc2c1cc1n(c2=O)Cc2c-1nc1cc(F)c(C)c3c1c2[C@H](OCNC(=O)CNC(=O)[C@H](Cc1ccccc1)NC(=O)CNC(=O)CNC(=O)CCCCCN1C(=O)C=CC1=O)CC3]

DMTMM (4.1 mg, 0.0148 mmol) was added to a solution of E-35a* (7.0 mg, 0.0134 mmol), N-methyl morpholine (2.9 μL, 0.0268 mmol) and MC-GGF-OH (CAS #1599440-15-9) (6.3 mg, 0.0134 mmol) in anhydrous DMF (2 mL) and the reaction was stirred at ambient temperature for 1 hour. The reaction was diluted with 5 mL of 1:1 acetonitrile/water (0.1% formic acid) and purified by C18 flash chromatography (gradient elution; 10% acetonitrile/water-100% acetonitrile) to afford compound E-39 (6.5 mg, 0.018 mmol, 50%) as a pale yellow solid. MS (ESI) m/z: 977.4 [M+H]⁺.

### Synthesis of E-40

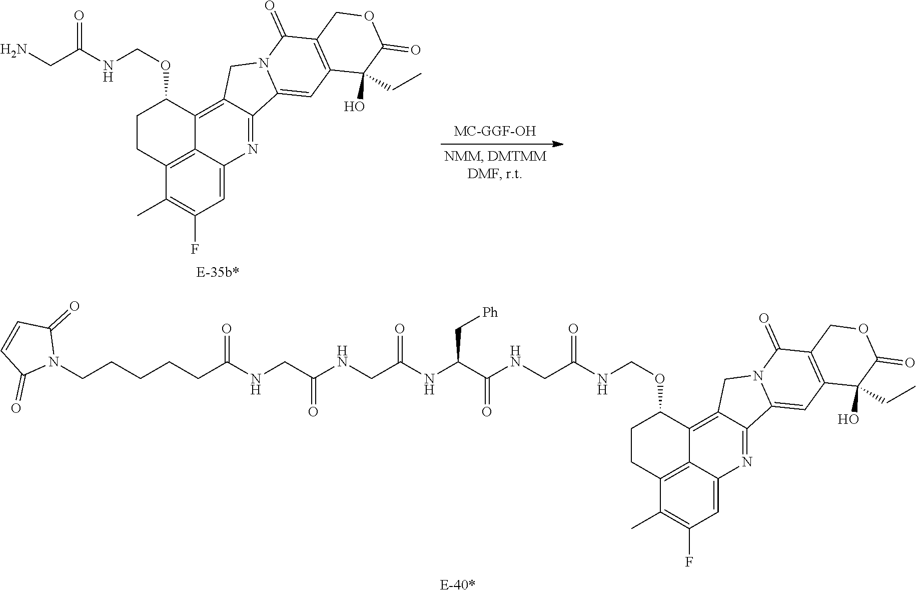 [SMILES: CC[C@@]1(O)C(=O)OCc2c1cc1n(c2=O)Cc2c-1nc1cc(F)c(C)c3c1c2[C@@H](OCNC(=O)CN)CC3 ; CC[C@@]1(O)C(=O)OCc2c1cc1n(c2=O)Cc2c-1nc1cc(F)c(C)c3c1c2[C@@H](OCNC(=O)CNC(=O)[C@H](Cc1ccccc1)NC(=O)CNC(=O)CNC(=O)CCCCCN1C(=O)C=CC1=O)CC3]

DMTMM (6.9 mg, 0.025 mmol) was added to a solution of E-35b* (12.0 mg, 0.023 mmol), N-methyl morpholine (4.9 μL, 0.046 mmol) and MC-GGF-OH (CAS #1599440-15-9) (10.8 mg, 0.023 mmol) in anhydrous DMF (3 mL) and the reaction was stirred at ambient temperature for 1 hour. The reaction was diluted with 5 mL of 1:1 acetonitrile/water (0.1% formic acid) and purified by C18 flash chromatography (gradient elution; 10% acetonitrile/water-100% acetonitrile) to afford compound E-40 (4.0 mg, 0.0041 mmol, 18%) as a pale yellow solid. MS (ESI) m/z: 977.4 [M+H]⁺.

### Synthesis of Compound E-19

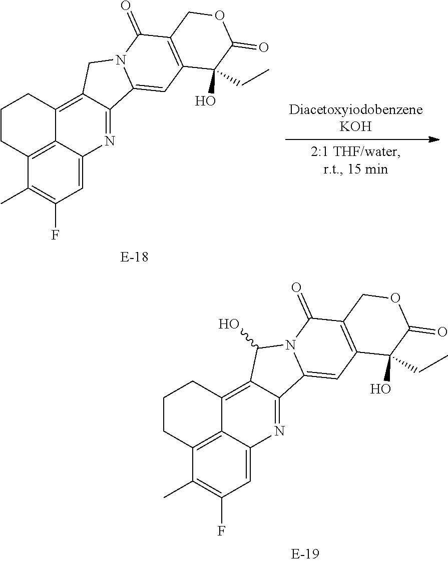 [SMILES: CC[C@@]1(O)C(=O)OCc2c1cc1n(c2=O)Cc2c-1nc1cc(F)c(C)c3c1c2CCC3 ; CC[C@@]1(O)C(=O)OCc2c1cc1n(c2=O)C(O)c2c-1nc1cc(F)c(C)c3c1c2CCC3]

Diacetoxyiodobenzene (8.5 mg, 0.026 mmol) was added to a solution of E-18 (10 mg, 0.024 mmol) and KOH (4 mg, 0.071 mmol) in 2:1 THF/water (1 mL). The reaction was stirred at ambient temperature for 15 min, then purified by reverse phase C18 flash chromatography (gradient elution; 30% ACN/water-100% ACN) to afford compound E-19 (4.1 mg, 0.0094 mmol, 39%) as a white solid. MS (ESI) m/z: 437.0 [M+H]⁺.

### Synthesis of Compounds E-20 and E-25

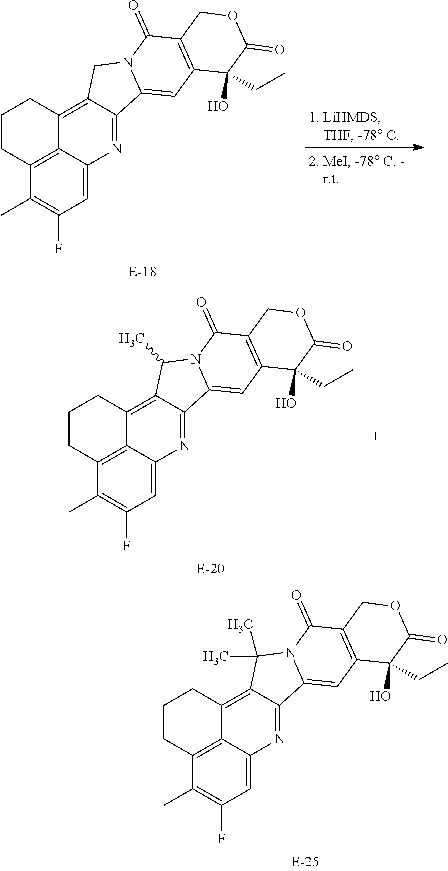

Lithium hexamethyldisilazide (LiHMDS) (1M in THF, 0.33 mL, 0.33 mmol) was added dropwise to a cloudy solution of E-18 (40 mg, 0.095 mmol) in THF (3 mL) at −78° C. Methyl iodide (12 μL, 0.19 mmol) was added and the solution was allowed to slowly warm to ambient temperature over 30 min. Reaction was partitioned between DCM (100 mL) and brine water (20 mL, pH=6-7). The organic layer was removed, and aqueous layer extracted again with DCM (50 mL). The combined organic layers were dried (Na₂SO₄), filtered, concentrated in vacuo, and purified by reverse phase C18 flash chromatography (gradient elution; 30% ACN/water—100% ACN) to afford compound E-20 (0.3 mg, 0.00069 mmol, 0.7%), and E-25 (2 mg, 0.0045 mmol, 5%) as white solids. Compound E-20: MS (ESI) m/z: 435.0 [M+H]⁺; Compound E-25: (ESI) m/z: 449.0 [M+H]⁺.

### Synthesis of Compounds E-21 and E-23

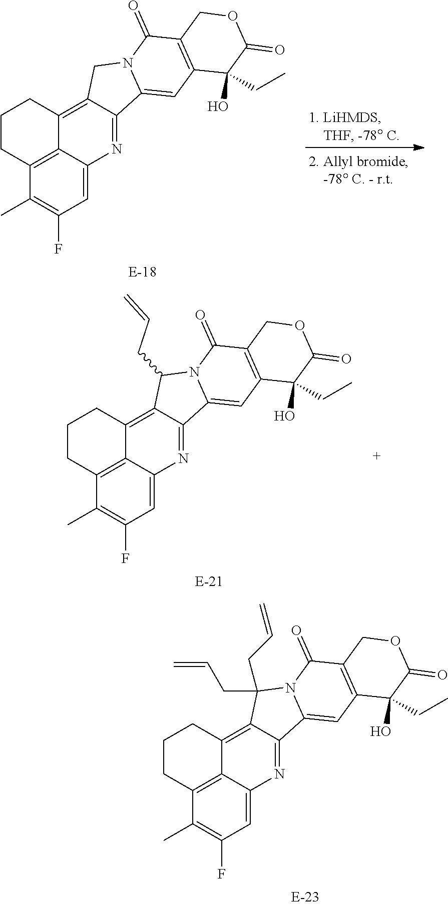

LiHMDS (1M in THF, 0.714 mL, 0.714 mmol) was added dropwise to a cloudy solution of E-18 (100 mg, 0.238 mmol) in THF (12 mL) at −78° C. Allyl bromide (41 μL, 0.476 mmol) was added and the solution was allowed to slowly warm to ambient temperature over 30 min. Reaction was partitioned between DCM (100 mL) and brine water (20 mL, pH=6-7). The organic layer was removed, and aqueous layer extracted again with DCM (50 mL). The combined organic layers were dried (Na₂SO₄), filtered, concentrated in vacuo, and purified by reverse phase C18 flash chromatography (gradient elution; 30% ACN/water-100% ACN) to afford compound E-21 (20 mg, 0.043 mmol, 18%), and E-23 (10 mg, 0.020 mmol, 8%) as white solids. Compound E-21: MS (ESI) m/z: 461.0 [M+H]⁺; Compound E-23: (ESI) m/z: 501.0 [M+H]⁺.

### Synthesis of Compound E-24

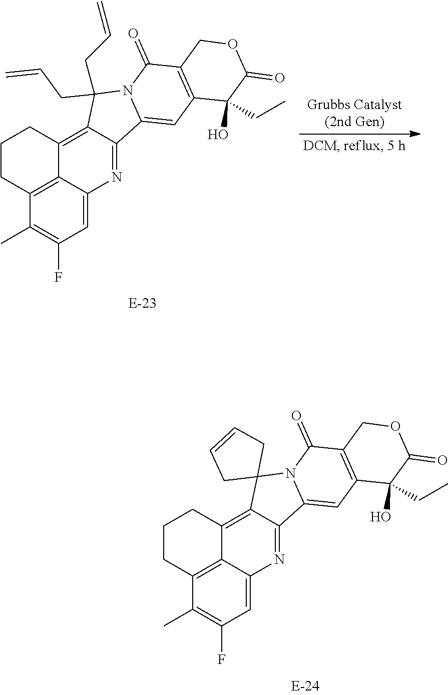

An oven dried round bottom flask was purged with dry N₂ (g) and charged with E-23 (10 mg, 0.020 mmol), second generation Grubbs Catalyst (1.7 mg, 0.002 mmol) and anhydrous DCM (2.5 mL). The reaction was refluxed for 5 h, cooled to ambient temperature, filtered through celite and concentrated in vacuo. The residue was purified by reverse phase C18 flash chromatography (gradient elution; 30% ACN/water-100% ACN) to afford compound E-24 (6.5 mg, 0.014 mmol, 69%) as a white solid. MS (ESI) m/z: 473.0 [M+H]⁺.

### Synthesis of Compound E-22

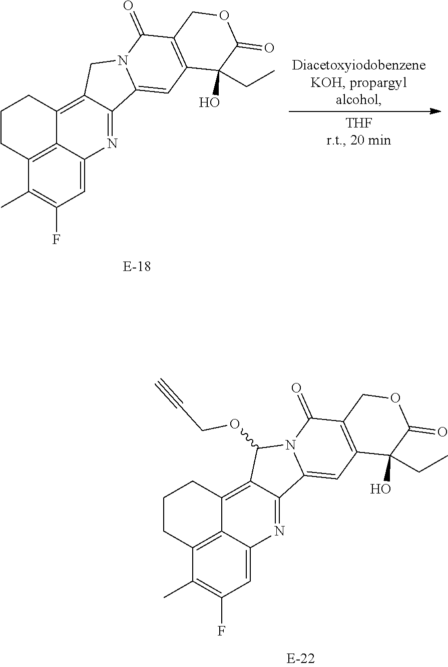

Diacetoxyiodobenzene (8.5 mg, 0.026 mmol) was added to a solution of E-18 (10 mg, 0.024 mmol), KOH (4 mg, 0.071 mmol) and propargyl alcohol (140 μL, 2.4 mmol) in anhydrous THF (1 mL). The reaction was stirred at ambient temperature for 20 min, then purified by reverse phase C18 flash chromatography (gradient elution; 25% ACN/water-100% ACN) to afford compound E-22 (2.0 mg, 0.0042 mmol, 18%) as a white solid. MS (ESI) m/z: 475.0 [M+H]⁺.

### Synthesis of Compound E-41

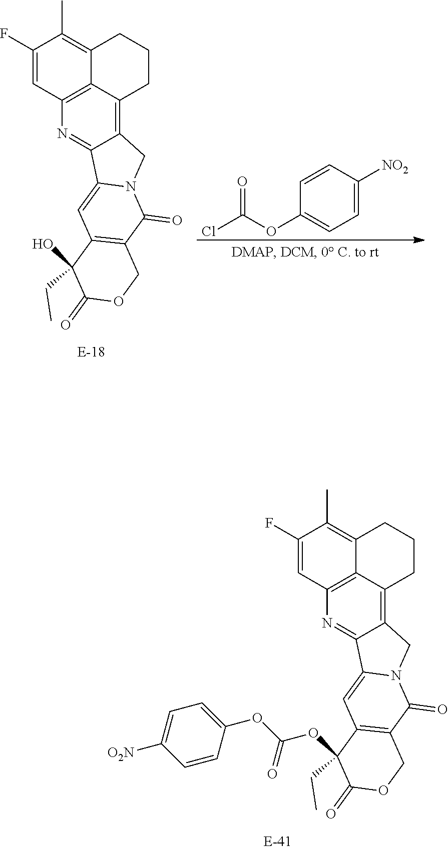

To a vial was added E-18 (100 mg, 0.2379 mmol), and dissolved in DCM (6.8 mL) at rt under Ar. DMAP (180 mg, 1.4747 mmol, 6.2 eq) was added to the vial and stirred for 3 min at rt, then cooled to 0° C. 4-Nitrophenyl chloroformate (172 mg, 0.8563 mmol, 3.6 eq) was dissolved in DCM (3.42 mL) and added dropwise to the vial containing E-18 dropwise. The vial was warmed to rt and stirred for 30 minutes. The crude reaction mixture was injected directly on an 80 g silica column with DCM to 5% MeOH/DCM gradient. The product fractions were combined and concentrated to obtain E-41 as a green solid (66.4 mg, 47% yield). MS (ESI) m/z: 586.28 [M+H]⁺.

### Synthesis of Compound E-43 and E-49

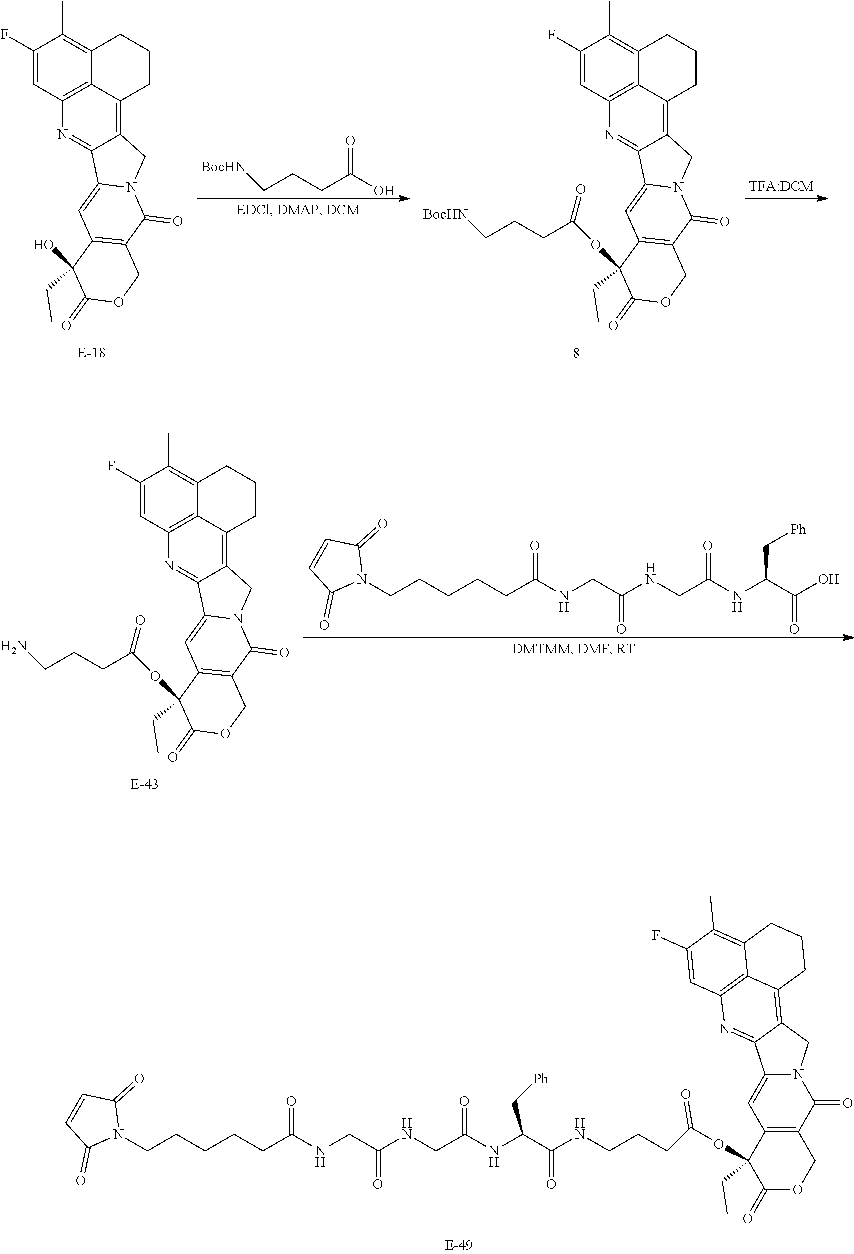 [SMILES: CC[C@@]1(O)C(=O)OCc2c1cc1n(c2=O)Cc2c-1nc1cc(F)c(C)c3c1c2CCC3 ; CC(C)(C)OC(=O)NCCCC(=O)O ; CC[C@@]1(OC(=O)CCCNC(=O)OC(C)(C)C)C(=O)OCc2c1cc1n(c2=O)Cc2c-1nc1cc(F)c(C)c3c1c2CCC3 ; CC[C@@]1(OC(=O)CCCN)C(=O)OCc2c1cc1n(c2=O)Cc2c-1nc1cc(F)c(C)c3c1c2CCC3 ; O=C(CCCCCN1C(=O)C=CC1=O)NCC(=O)NCC(=O)N[C@@H](Cc1ccccc1)C(=O)O ; CC[C@@]1(OC(=O)CCCNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3]

### Synthesis of Compound 8

To the solution of E-18 (0.010 g, 0.024 mmol), N-Boc-butyric acid (0.0145 g, 0.071 mmol), and DMAP (0.0017 g, 0.014 mmol) in anhydrous DCM under Argon at 0° C., EDCI (0.0137 g, 0.071 mmol) was added and stirred overnight. LCMS after 22 h showed almost 80% product conversion. Then an additional 1 eq of EDCI and 1 eq of N-Boc butyric acid was added to the reaction mixture. After 3 h, diluted with DCM and washed with water. The aqueous layer extracted with DCM. The combined organic layer dried over sodium sulfate and evaporated. The crude was purified with normal phase using DCM/MeOH (0 to 20% MeOH in DCM gradient to get the product 8 (0.008 g, 0.014 mmol, 53% yield). MS (ESI) m/z: 606.05 [M+H]⁺.

### Synthesis of Compound E-43

To the solution of 8 (0.008 g, 0.014 mmol) in anhydrous DCM (1 ml) under Argon at RT, Trifluoroacetic acid (1 ml) was added. After 30 minutes, solvent evaporated and residual oil co-evaporated with ether (4*2 ml) to get the white residue (0.011 g, 0.018 mmol, quantitative yield). MS (ESI) m/z: 506.3 [M+H]⁺.

### Synthesis of Compound E-49

To the mixture of E-43 (0.011 g, 0.018 mmol), MC-GGF-OH (0.0105 g, 0.022 mmol), and DMTMM (0.0098 g, 0.036 mmol) under Argon at RT, anhydrous DMF (1.1 ml) was added and stirred at RT until completion. The LCMS showed the reaction was not complete in 1 h. Then N-methyl morpholine (0.002 ml, 0.018 mmol) and MC-GGF-OH (0.008 g, 0.018 mmol) was added to the reaction mixture. After 30 minutes, directly purified with reverse phase (Gemini) using water/Acetonitrile (neutral) gradient. The product containing fractions combined and lyophilized to get the white solid (0.0042 g, 0.004 mmol, 25% yield). MS (ESI) m/z: 960.4 [M+H]⁺.

### Synthesis of E-44

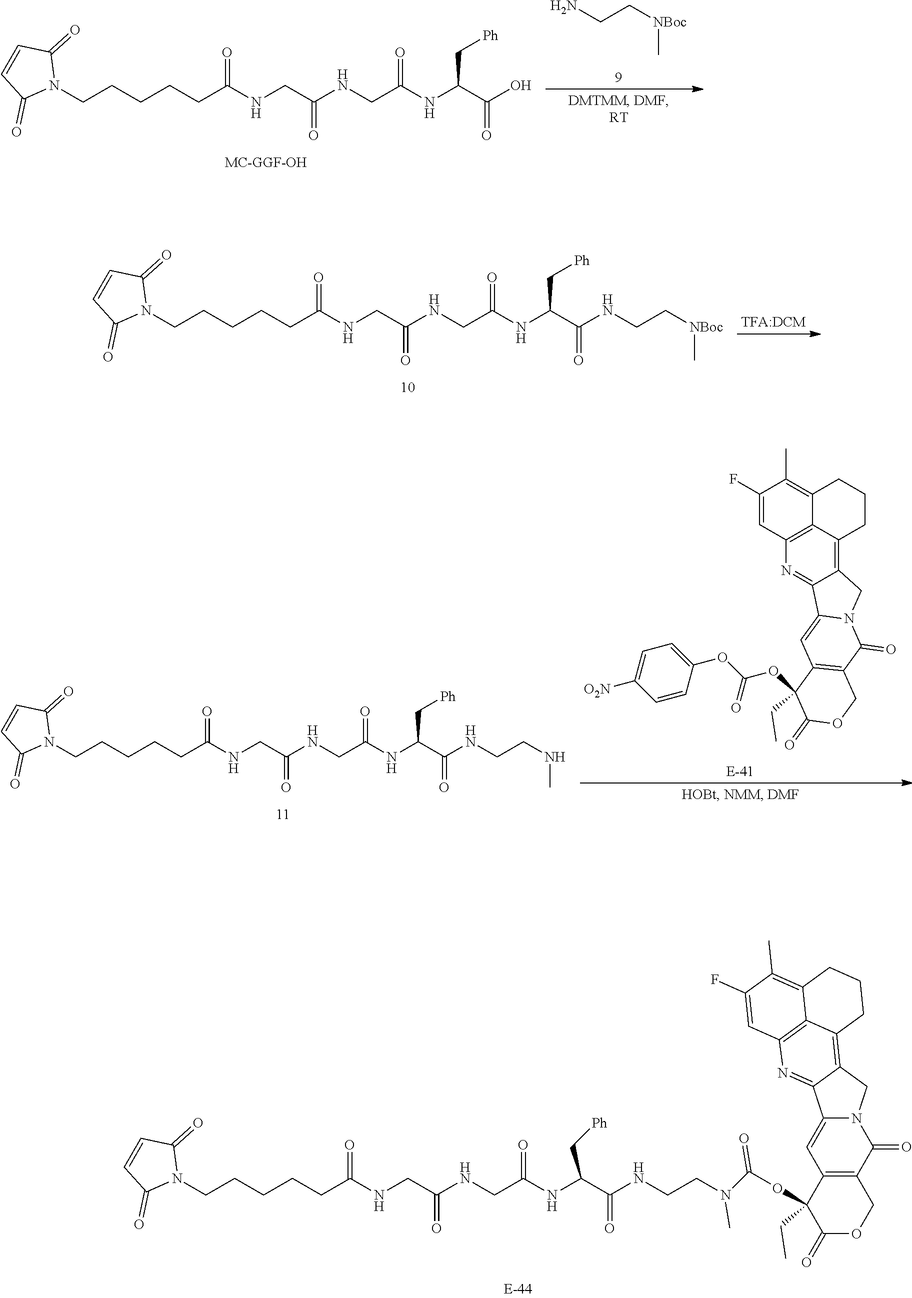 [SMILES: O=C(CCCCCN1C(=O)C=CC1=O)NCC(=O)NCC(=O)N[C@@H](Cc1ccccc1)C(=O)O ; CN(CCN)C(=O)OC(C)(C)C ; CN(CCNC(=O)[C@H](Cc1ccccc1)NC(=O)CNC(=O)CNC(=O)CCCCCN1C(=O)C=CC1=O)C(=O)OC(C)(C)C ; CNCCNC(=O)[C@H](Cc1ccccc1)NC(=O)CNC(=O)CNC(=O)CCCCCN1C(=O)C=CC1=O ; CC[C@@]1(OC(=O)Oc2ccc([N+](=O)[O-])cc2)C(=O)OCc2c1cc1n(c2=O)Cc2c-1nc1cc(F)c(C)c3c1c2CCC3 ; CC[C@@]1(OC(=O)N(C)CCNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3]

### Synthesis of Compound 10

To the solution of MC-GGF-OH (0.100 g, 0.212 mmol), and 1-Boc-1 methylethylenediamine (0.076 ml, 0.423 mmol) in anhydrous DMF (0.07M) under Argon at RT, DMTMM (0.117 g, 0.423 mmol) was added and stirred at RT until completion. After 30 min, the reaction mixture was purified directly with Reverse phase (C18Aq, 150 g) using water/acetonitrile (neutral). The product containing fractions combined and lyophilized to get 10 (0.074 g, 0.118 mmol, 51% yield) as white solid. MS (ESI) m/z: 629.4 [M+H]⁺.

### Synthesis of Compound 11

To the solution of 10 (0.074 g, 0.117 mmol) in anhydrous DCM at RT, TFA was added and stirred at RT until completion of the reaction. After 30 min, solvent evaporated and crude oil co-evaporated with ether (4*3 ml) and kept in high vacuum. The compound purified with reverse phase (C18Aq, 50 g) using water/acetonitrile (0.1% TFA) to get the white solid (0.058 g, 77% yield) as a TFA salt. MS (ESI) m/z: 529.3 [M+H]⁺.

### Synthesis of Compound E-44

To the solution of E-41 (0.040 g, 0.068 mmol) and 5a (0.054 g, 0.102 mmol) in anhydrous DMF (0.035M) under Argon at RT, HOBt hydrate (0.010 g, 0.068 mmol) and N-methyl morpholine (0.011 ml, 0.102 mmol) were added. As the reaction was 80% complete in 4 h, purified directly with reverse phase (Gemini) using water/acetonitrile (neutral). The product repurified through Gemini to get product as white solid (0.017 g, 0.017 mmol, 25.2% yield). MS (ESI) m/z: 975.3 [M+H]⁺.

### Synthesis of E-45 and E-46

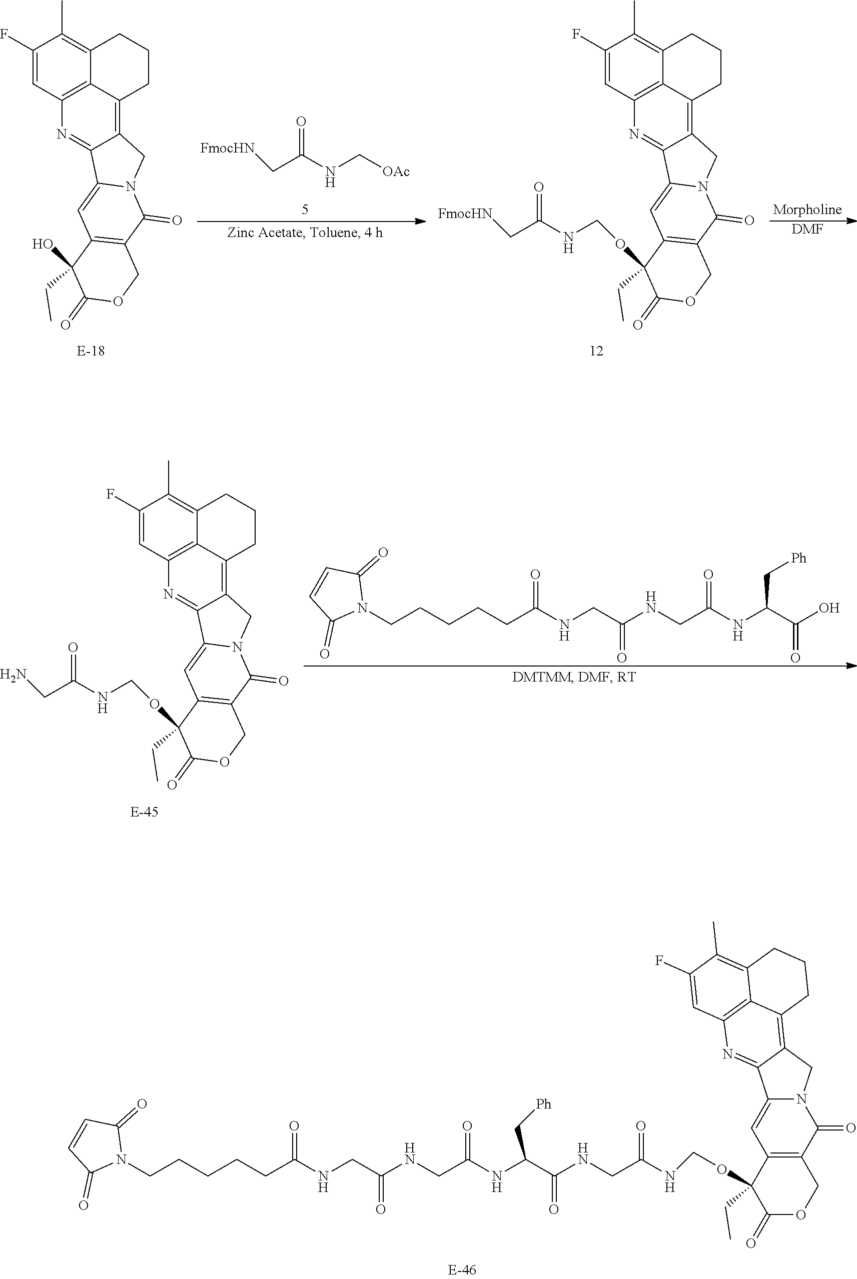

### Synthesis of Compound 12

To the mixture of E-18 (0.068 g, 0.163 mmol), 5 (0.030 g, 0.081 mmol), and Zinc acetate (0.0149 g, 0.081 mmol), anhydrous toluene (5 ml) was added under Argon and refluxed at 115° C. for 4 h. Similar reaction was set up with E-18 (0.039 g, 0.092 mmol), 3 (0.017 g, 0.046 mmol), and Zinc acetate (0.0085 g, 0.046 mmol) in anhydrous toluene (2 ml). After 4 h, combined, solvent evaporated and purified with normal phase using DCM/MeOH (0-20% gradient) followed by reverse phase (Gemini) using water/Acetonitrile (neutral) to get the product 12 (0.0016 g, 0.002 mmol, 1.7% yield) as an off-white solid. MS (ESI) m/z: 729.3 [M+H]⁺.

### Synthesis of Compound E-45

To the solution of 12 (0.0016 g, 0.002 mmol) in anhydrous DMF at RT under Argon, solution of Morpholine in DMF (1:4) (0.040 ml) was added. After 2 h, diluted with cold 1 ml diethyl ether and kept in −20° C. overnight. Then centrifuged (7500 RPM, 2° C., 5 min), supernatant decanted and solid washed with another 1 ml of cold diethyl ether. The suspension vortexed, centrifuged and the supernatant decanted. The white solid kept in high vacuum overnight to get E-45 (0.001 mg, 0.0017 mmol, 82% yield). MS (ESI) m/z: 507.3 [M+H]⁺.

### Synthesis of Compound E-46

To the mixture of E-45 (0.001 g, 0.0017 mmol), MC-GGF-OH (0.001 g, 0.002 mmol), and DMTMM (0.001 g, 0.004 mmol) under Argon at RT, anhydrous DMF (0.090 ml) was added and stirred at RT until completion. After 30 min, diluted with water/Acetonitrile and purified with Reverse phase (Gemini) using water/acetonitrile (neutral). The product containing fraction combined and lyophilized to get E-46 as a white solid (0.0013 g, 0.0013 mmol, 77% yield). MS (ESI) m/z: 961.4 [M+H]⁺.

### Synthesis of E-47

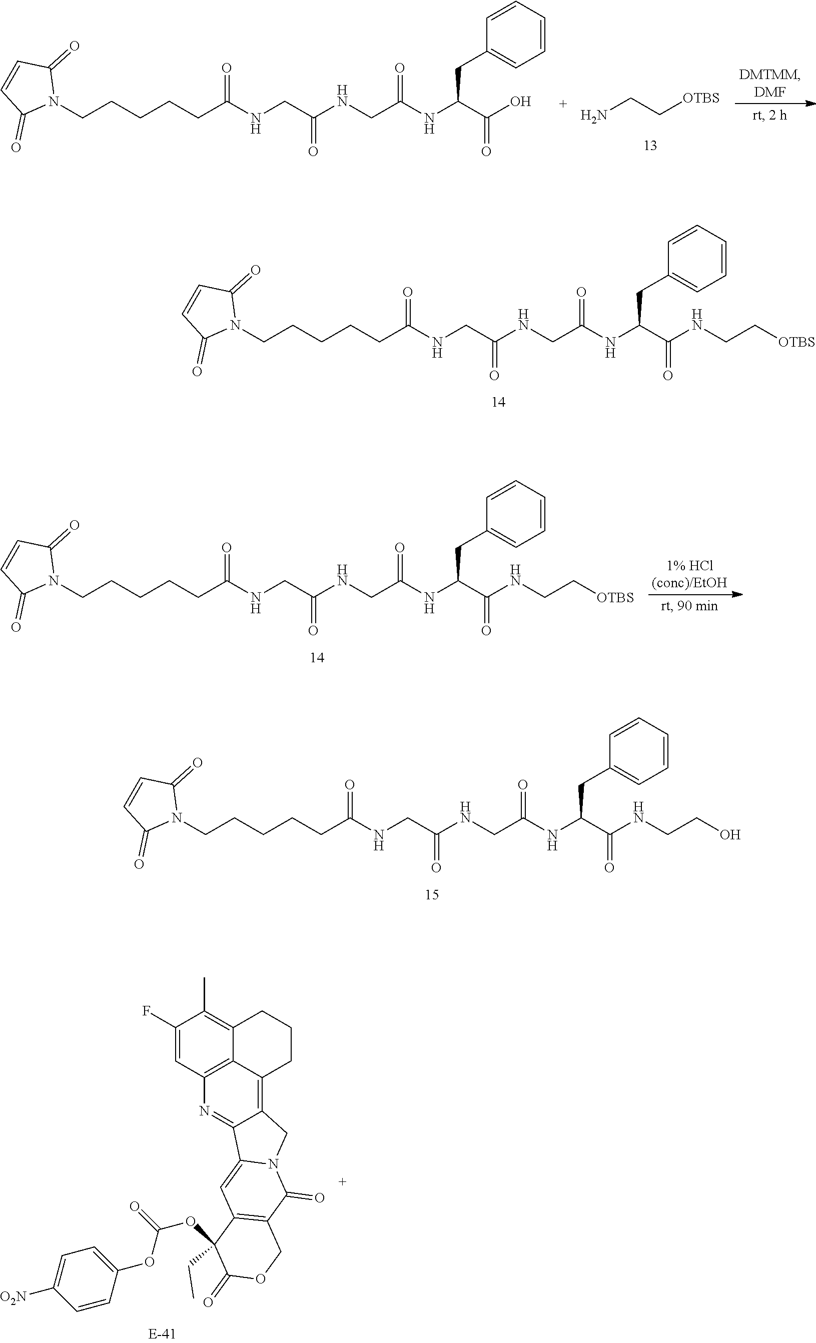 [SMILES: O=C(CCCCCN1C(=O)C=CC1=O)NCC(=O)NCC(=O)N[C@@H](Cc1ccccc1)C(=O)O ; CC(C)(C)[Si](C)(C)OCCN ; CC(C)(C)[Si](C)(C)OCCNC(=O)[C@H](Cc1ccccc1)NC(=O)CNC(=O)CNC(=O)CCCCCN1C(=O)C=CC1=O ; CC(C)(C)[Si](C)(C)OCCNC(=O)[C@H](Cc1ccccc1)NC(=O)CNC(=O)CNC(=O)CCCCCN1C(=O)C=CC1=O ; O=C(CCCCCN1C(=O)C=CC1=O)NCC(=O)NCC(=O)N[C@@H](Cc1ccccc1)C(=O)NCCO ; CC[C@@]1(OC(=O)Oc2ccc([N+](=O)[O-])cc2)C(=O)OCc2c1cc1n(c2=O)Cc2c-1nc1cc(F)c(C)c3c1c2CCC3]

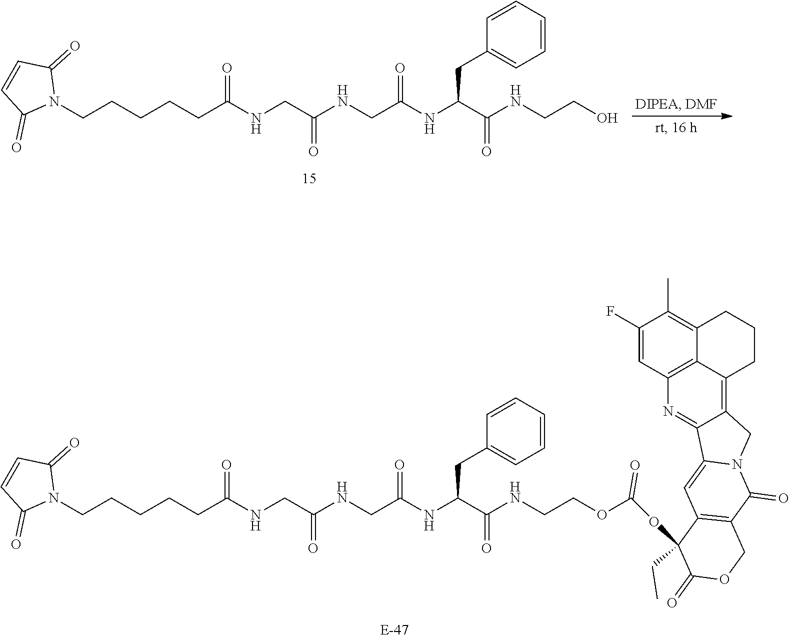 [SMILES: O=C(CCCCCN1C(=O)C=CC1=O)NCC(=O)NCC(=O)N[C@@H](Cc1ccccc1)C(=O)NCCO ; CC[C@@]1(OC(=O)OCCNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3]

### Synthesis of Compound 14

To a vial was added MC-GGF-OH (100 mg, 0.212 mmol) and dissolved in DMF (2.2 mL) at rt under Ar. To the vial was added 13 (0.055 mL, 0.2646 mmol) and stirred for 5 minutes at rt. To the vial was added DMTMM (118 mg, 0.4233 mmol) and the reaction was stirred for 1 h. The crude reaction was diluted with (1:1) (H₂O:ACN) (+0.05% FA) (1 mL) and purified on 50 g C18 with a gradient of (5% ACN/H₂O) (+0.05% FA) to (95% ACN/H₂O) (+0.05% FA). The product fractions were combined and lyophilized to give 14 as a white solid (51.6 mg, 39% yield). (ESI) m/z: 630.67 [M+H]⁺.

### Synthesis of Compound 15

To a vial was added 14 (5 mg, 0.008 mmol) and dissolved in 1% HCl (conc.)/EtOH (0.159 mL). The vial was stirred at rt for 90 minutes until all 14 was consumed. The reaction was concentrated and 15 was used as is in the next step. (ESI) m/z: 630.67 [M+H]⁺.

### Synthesis of E-47

To a vial was added E-41 (3 mg, 0.0051 mmol) and 15 (4 mg, 0.0077 mmol) and dissolved in DMF (0.15 mL) at rt under Ar. To the vial was added DIPEA (0.001 mL, 0.0077 mmol) and stirred at rt for 1 h. At 1 h, HOBt (4 mg, 0.0204 mmol) was added and the reaction was stirred at rt for 16 h. The crude reaction was diluted with (1:1) (H₂O:ACN) (+0.05% FA) (1 mL) and purified with reverse phase (Gemini column) with a gradient of (5% ACN/H₂O) (+0.05% FA) to (95% ACN/H₂O) (+0.05% FA). The product fractions were lyophilized to obtain E-47 as a white solid (0.4 mg, 8% yield). (ESI) m/z: 962.35 [M+H]⁺.

### Synthesis of E-42 and E-48

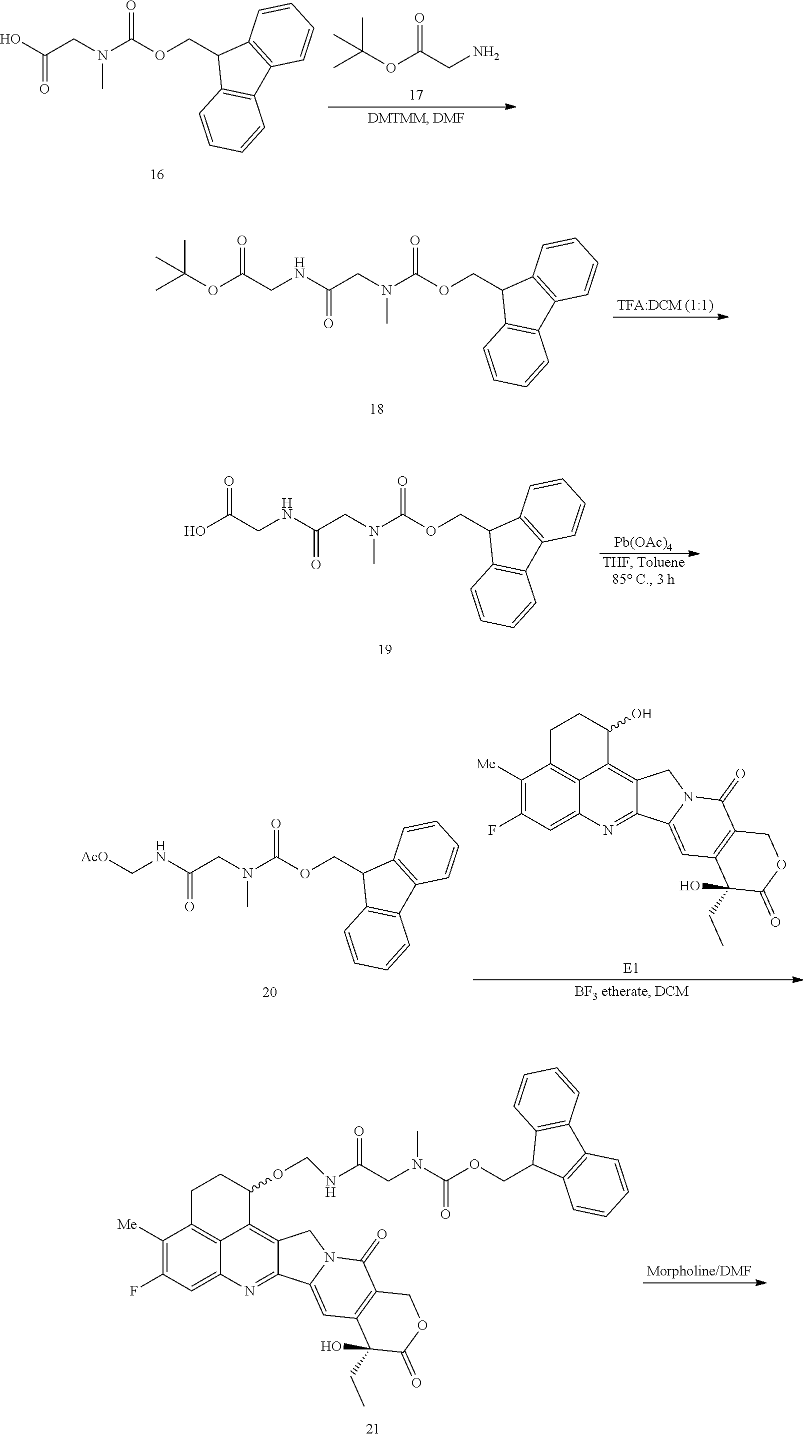

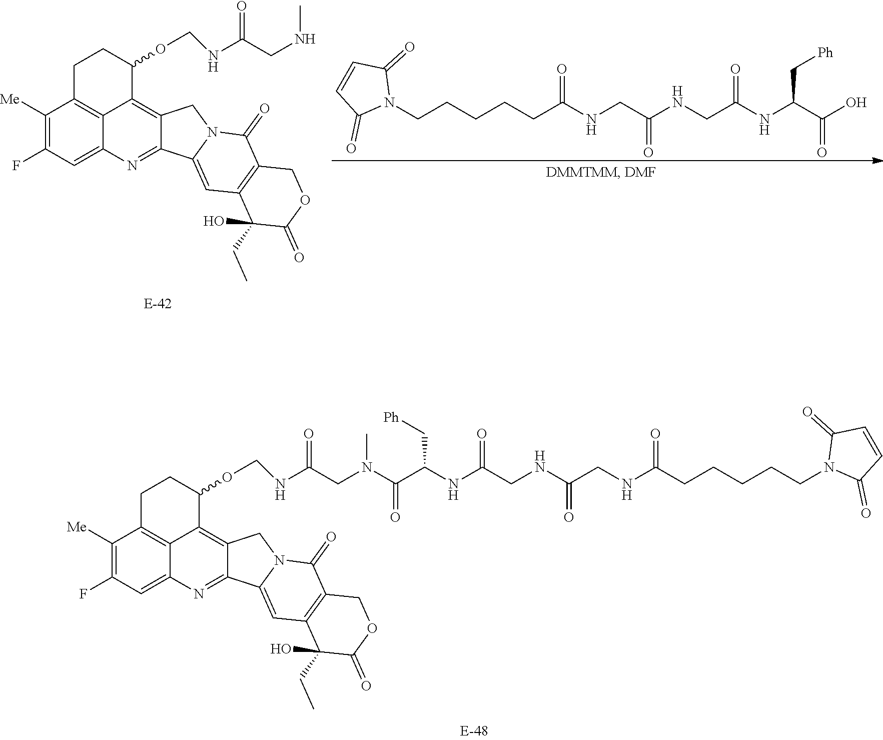 [SMILES: CC[C@@]1(O)C(=O)OCc2c1cc1n(c2=O)Cc2c-1nc1cc(F)c(C)c3c1c2C(OCNC(=O)CNC)CC3 ; O=C(CCCCCN1C(=O)C=CC1=O)NCC(=O)NCC(=O)N[C@@H](Cc1ccccc1)C(=O)O ; CC[C@@]1(O)C(=O)OCc2c1cc1n(c2=O)Cc2c-1nc1cc(F)c(C)c3c1c2C(OCNC(=O)CN(C)C(=O)[C@H](Cc1ccccc1)NC(=O)CNC(=O)CNC(=O)CCCCCN1C(=O)C=CC1=O)CC3]

### Synthesis of Compound 18

To the solution of 16 (0.500 g, 1.606 mmol) and 17 (0.263 g, 2.00 mmol) in anhydrous DMF under Argon at RT, DMTMM (0.889 g, 3.21 mmol) was added and stirred at RT until completion. After 2 h, water was added to quench the reaction and purified with reverse phase (C18, 100 g) using water/acetonitrile (neutral). The product containing fractions combined and lyophilized to get the product 18 (0.593 g, 1.39 mmol, 87% yield) as a white solid. MS (ESI) m/z: 425.2 [M+H]⁺.

### Synthesis of Compound 19

To the solution of 18 (0.593 g, 1.396 mmol) in anhydrous DCM (10 ml) at RT, TFA (10 ml) was added and stirred at RT until completion. After 1 h, solvent evaporated and the crude oil co-evaporated with ether (4*3 ml) and kept in high vacuum to get the white solid 19 (0.501 g, 1.36 mmol, 97% yield) which was taken to next step without further purification. MS (ESI) m/z: 369.1 [M+H]⁺.

### Synthesis of Compound 20

To the solution of compound 19 (0.501 g, 1.36 mmol) in anhydrous toluene (19 ml) and anhydrous THF (6 ml) at RT under Argon, Pb(OAc)₄ was added and transferred to 85° C. oil bath and refluxed for 3 h. After 3 h, the reaction mixture was brought to RT and filtered to remove the lead acetate adduct. The solid washed with anhydrous toluene and THF. The filtrate evaporated and the crude purified with normal phase (120 g column) using hexane/EtOAc (0-100% gradient) to get 20 (0.314 g, 0.821 mmol, 60% yield) as white solid. MS (ESI) m/z: 405.2 [M+Na]⁺.

### Synthesis of Compound 21

To the suspension of E-1 (0.040 g, 0.092 mmol) and 20 (0.070 g, 0.183 mmol) in anhydrous DCM at 0° C., BF3 etherate was added dropwise and stirred at 0° C. until completion. After 1 h, the reaction stalled. The solvent was evaporated. The crude was dissolved in DMF and purified with reverse phase (C18Aq, 50 g) using water/acetonitrile (neutral). The product containing fractions were combined and evaporated to get the product 21 (0.0122 g, 0.016 mmol, 18% yield) as a yellowish solid. MS (ESI) m/z: 759.2 [M+H]⁺.

### Synthesis of E-42

To 21 (0.012 g, 0.016 mmol) under Argon at RT, freshly prepared solution of Morpholine in DMF (1:4) (0.055M) was added and stirred at RT until completion. After 1 h, LCMS showed completion of the reaction. The cold ether was added to the reaction and kept at −20° C. for 1 h and then centrifuged (7500 RPM, 2° C., 5 min). The filtrate was decanted and the solid washed again with cold ether and centrifuged again to get the 0.0018 g of the product which was taken to next step without further purification. The filtrate combined and evaporated to remove the ether and purified with reverse phase (Gemini) using water/acetonitrile (neutral) to get E-42 (0.0034 g, 0.006 mmol, 38% yield). MS (ESI) m/z: 537.2 [M+H]⁺.

### Synthesis of E-48

To the solution of E-42 (0.0018 g, 0.003 mmol) and MC-GGF-OH (0.002 g, 0.004 mmol) in anhydrous DMF under Argon at RT, DMTMM (0.002 g, 0.007 mmol) was added and stirred at RT until completion. LCMS after 30 min showed completion of reaction. The reaction mixture directly purified with reverse phase (Gemini) using water/acetonitrile (neutral) to get the product (0.0011 g, 33% yield) as white solid. MS (ESI) m/z: 991.3 [M+H]⁺.

### Synthesis of Compound E-29

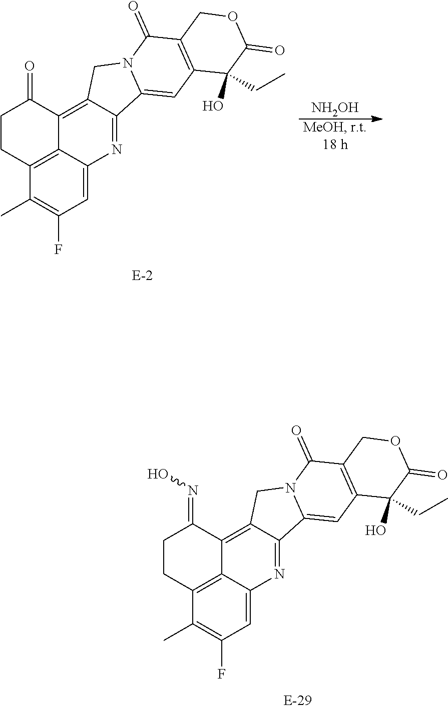 [SMILES: CC[C@@]1(O)C(=O)OCc2c1cc1n(c2=O)Cc2c-1nc1cc(F)c(C)c3c1c2C(=O)CC3 ; "NH2OH" ; "MeOH, r.t.18 h" ; CC[C@@]1(O)C(=O)OCc2c1cc1n(c2=O)Cc2c-1nc1cc(F)c(C)c3c1c2C(=NO)CC3]

Hydroxylamine (11 mg, 0.35 mmol) was added to a solution of E-2 (15 mg, 0.035 mmol) in MeOH (10 mL) and the reaction was stirred at ambient temperature for 18 h. The solvent was removed, and the resulting residue was dissolved in 2:1 ACN/water (3 mL), filtered, and filtrate purified by reverse phase C18 flash chromatography (gradient elution; 30% ACN/water-100% ACN) to afford compound E-29 (0.5 mg, 0.0011 mmol, 3%) as a white solid. MS (ESI) m/z: 450.0 [M+H]⁺.

### Synthesis of E-38

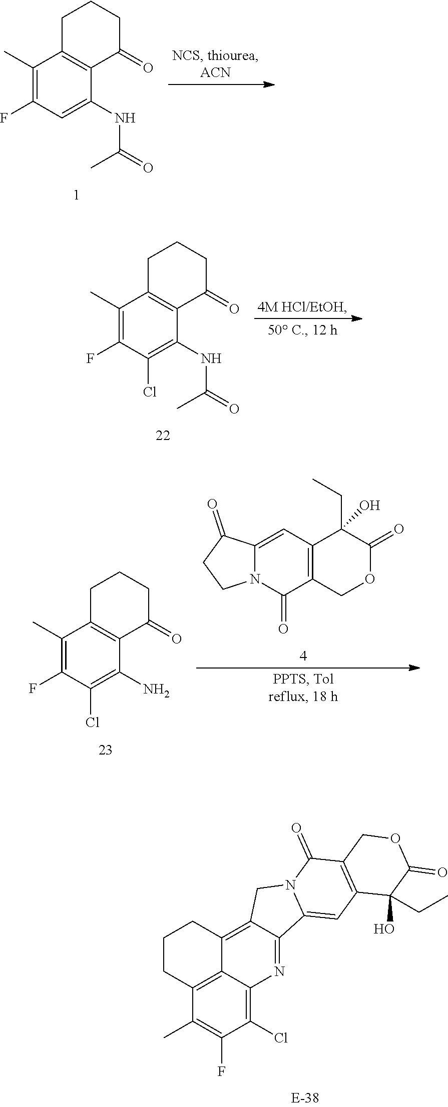 [SMILES: CC(=O)Nc1cc(F)c(C)c2c1C(=O)CCC2 ; CC(=O)Nc1c(Cl)c(F)c(C)c2c1C(=O)CCC2 ; Cc1c(F)c(Cl)c(N)c2c1CCCC2=O ; CC[C@@]1(O)C(=O)OCc2c1cc1n(c2=O)CCC1=O ; CC[C@@]1(O)C(=O)OCc2c1cc1n(c2=O)Cc2c-1nc1c(Cl)c(F)c(C)c3c1c2CCC3]

### Synthesis of Compound 22

Compound 1 (CAS #143655-58-7, 100 mg, 0.435 mmol) was dissolved in anhydrous acetonitrile (2.5 mL). This solution was then added to a mixture of N-chlorosuccinimide (NCS, 133 mg, 0.638, 1.5 eq) and thiourea (10 mg, 0.128, 0.3 eq) in anhydrous acetonitrile (2.5 mL) and stirred at ambient temperature for 90 min. The reaction mixture was then concentrated in vacuo, and the residue was purified by reverse phase C18 flash chromatography (gradient elution; 15% ACN/water-100% ACN) to afford compound 22 (41 mg, 0.152 mmol, 35%) as an off-white solid. MS (ESI) m/z: 297.95 [M+H]⁺.

### Synthesis of Compound 23

Compound 22 (56 mg, 0.208 mmol) was dissolved in 4M HCl in EtOH (5.0 mL) and stirred at 50° C. overnight. The reaction was cooled to ambient temperature, concentrated in vacuo, and the residue was purified by reverse phase C18 flash chromatography (gradient elution; 15% ACN/water-100% ACN) to afford compound 23 (40 mg, 0.176 mmol, 84%) as a light brown solid. MS (ESI) m/z: 228.00 [M+H]⁺.

### Synthesis of E-38

An oven-dried reaction vessel equipped with reflux condenser was charged with compound 23 (38 mg, 0.167 mmol), compound 4 (CAS #110351-94-5, 53 mg, 0.200 mmol, 1.2 eq), PPTS (2 mg, 0.008 mmol, 0.05 eq) and anhydrous toluene (10 mL). The mixture was refluxed for 48 h under atmosphere of nitrogen. The reaction was concentrated in vacuo, and the residue was triturated with acetonitrile. The crude solid was purified by silica gel flash chromatography (gradient elution; 100% DCM-20% MeOH/DCM) to afford compound E-38 (10 mg, 0.022 mmol, 13%) as a brown solid. MS (ESI) m/z: 455.13 [M+H]⁺.

### Proposed Synthesis E-3, E-4, E-32

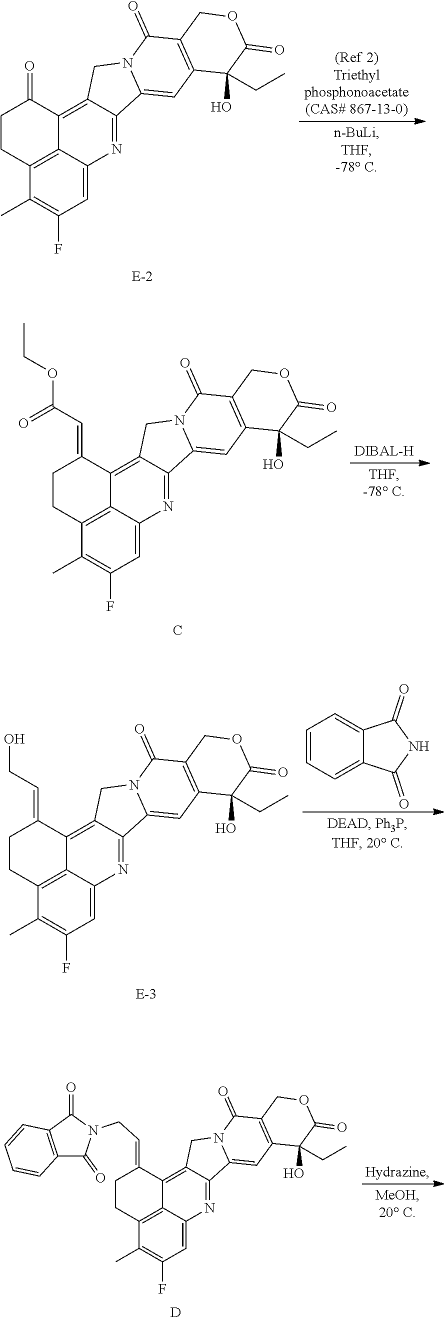

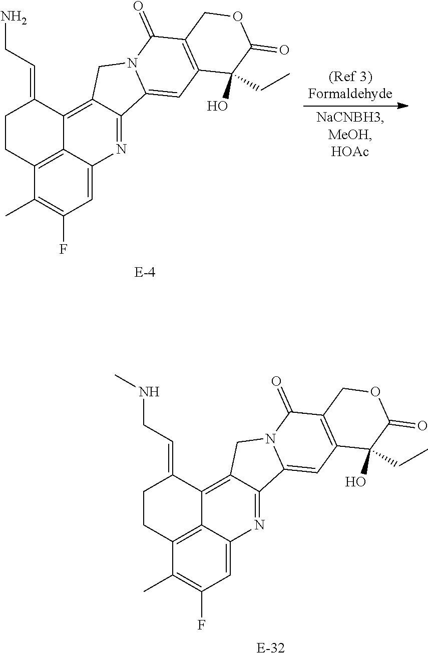

### Proposed Synthesis E-31, E-33, E-34

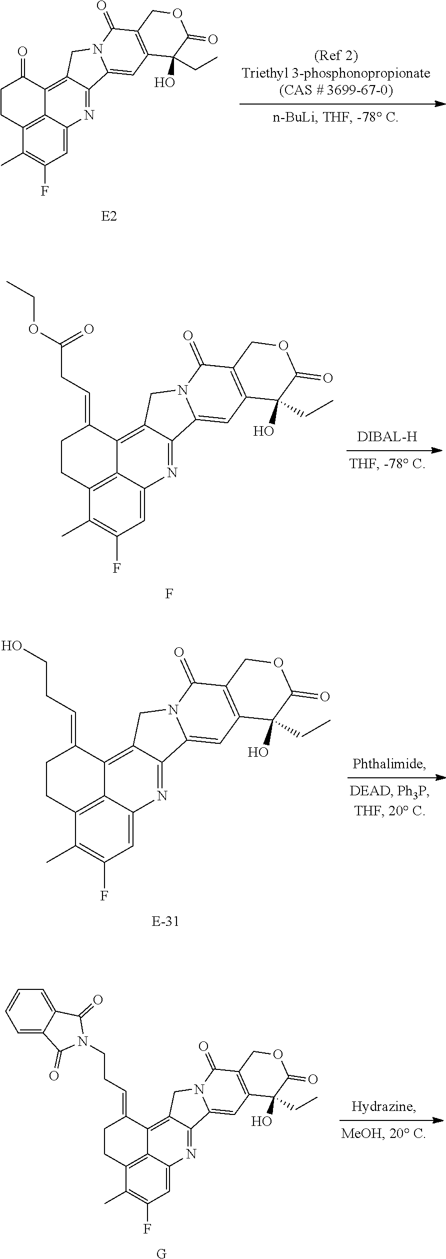

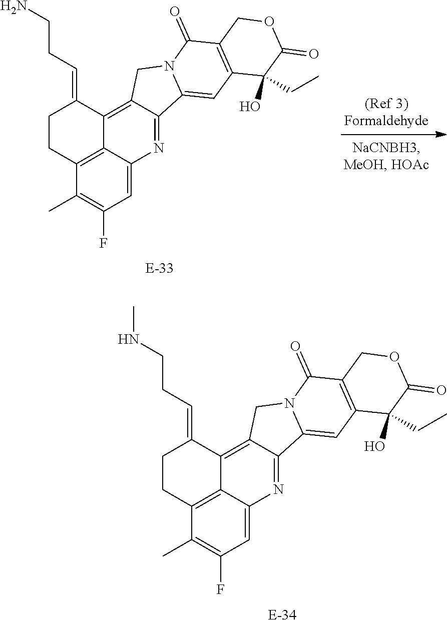

### Proposed Synthesis E-27

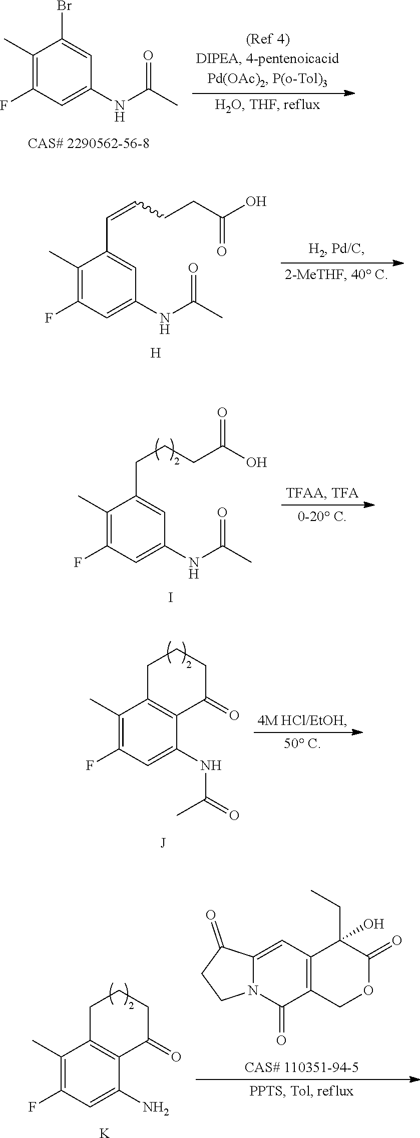

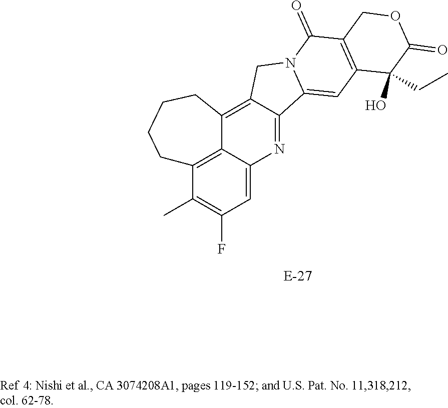 [SMILES: CC[C@@]1(O)C(=O)OCc2c1cc1n(c2=O)Cc2c-1nc1cc(F)c(C)c3c1c2CCCC3]

### Proposed Synthesis E-5

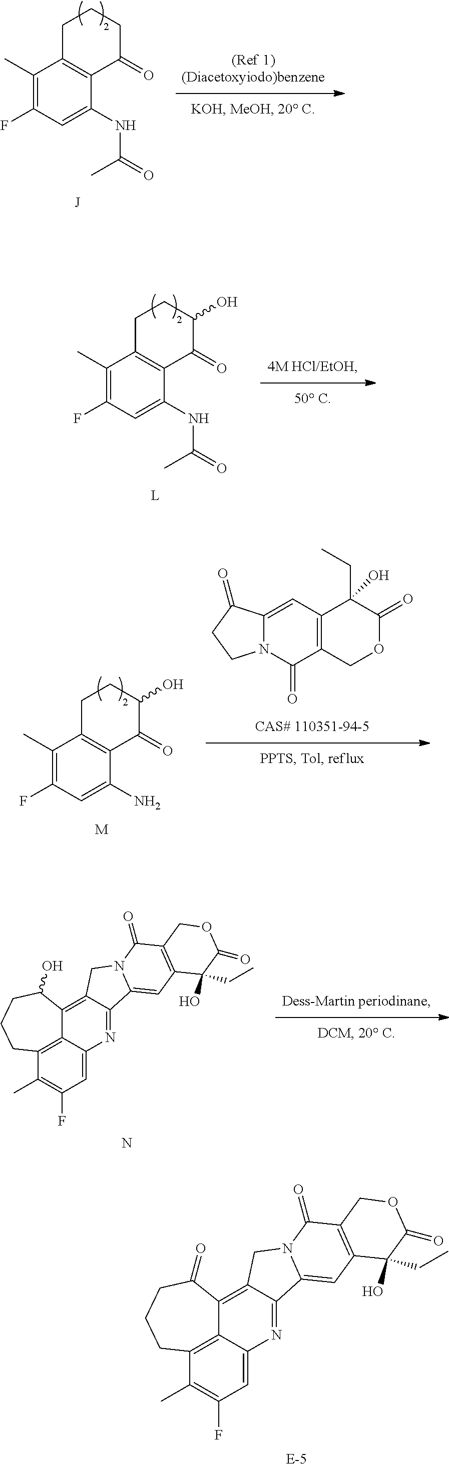

### Proposed Synthesis E-6

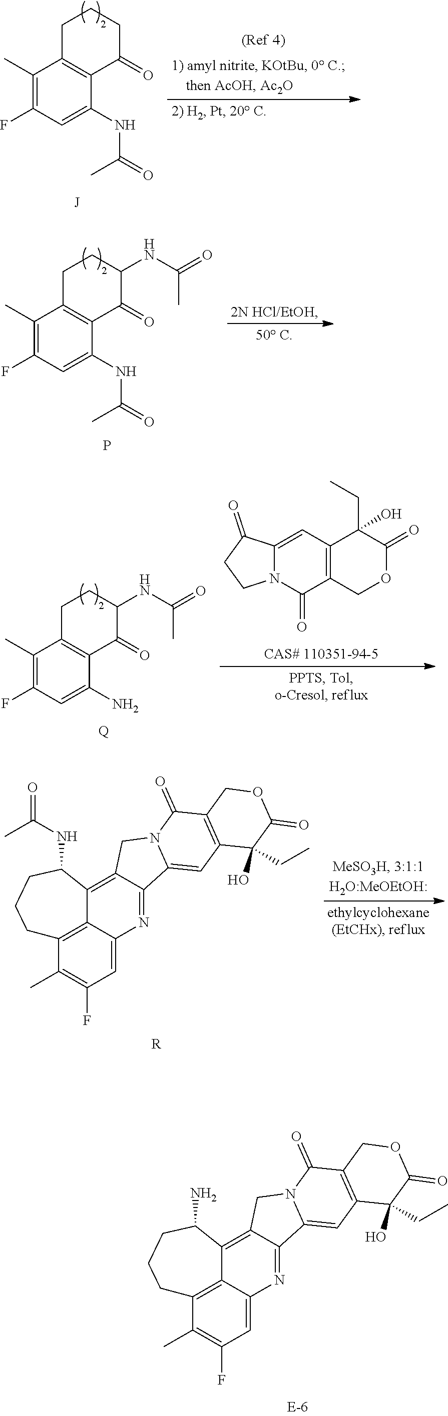

### Proposed Synthesis E-9

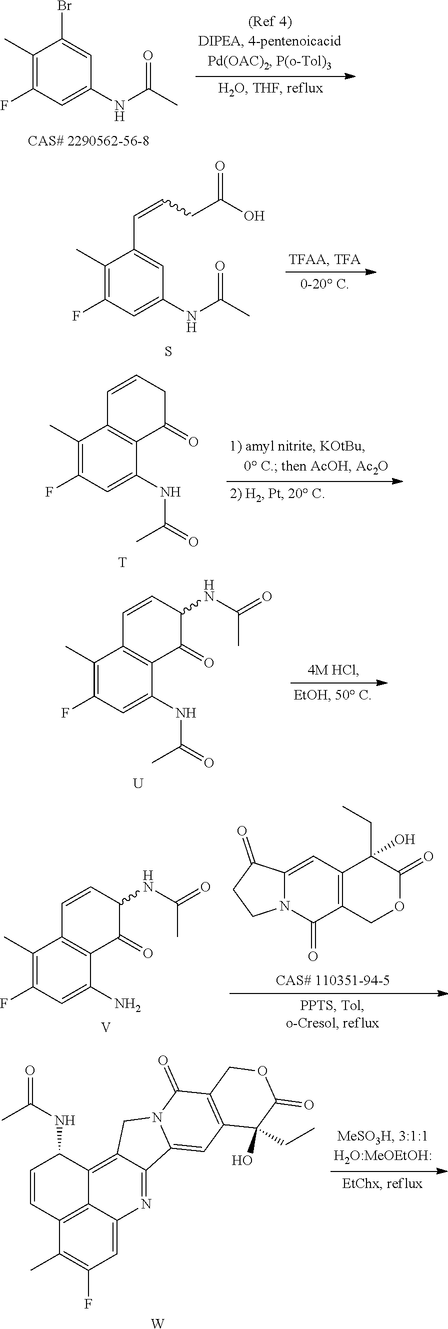

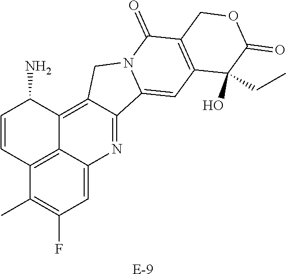 [SMILES: CC[C@@]1(O)C(=O)OCc2c1cc1n(c2=O)Cc2c-1nc1cc(F)c(C)c3c1c2[C@@H](N)C=C3 ; "E-9"]

### Proposed Synthesis E-11

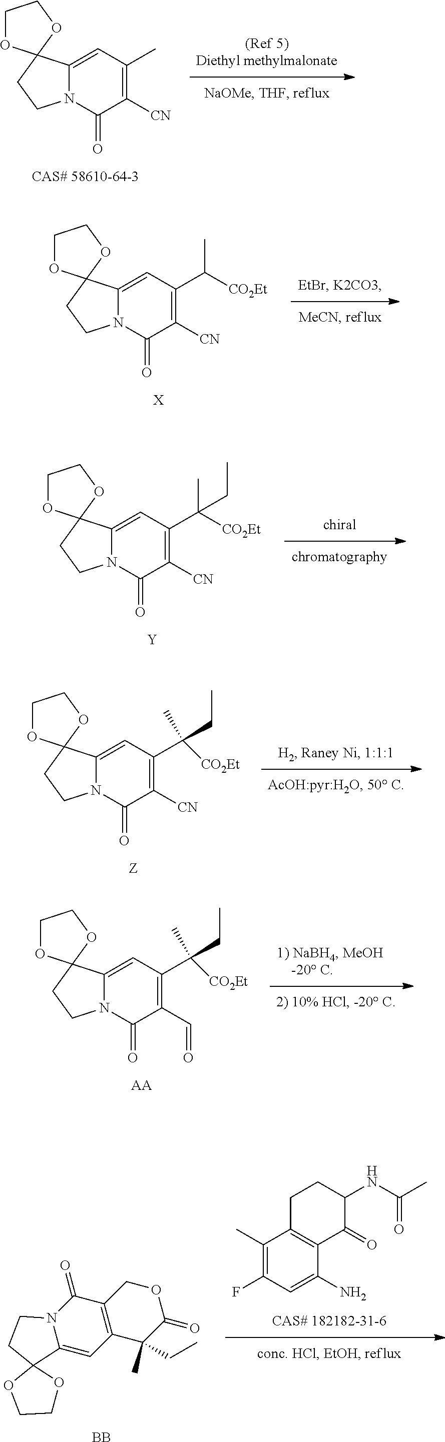

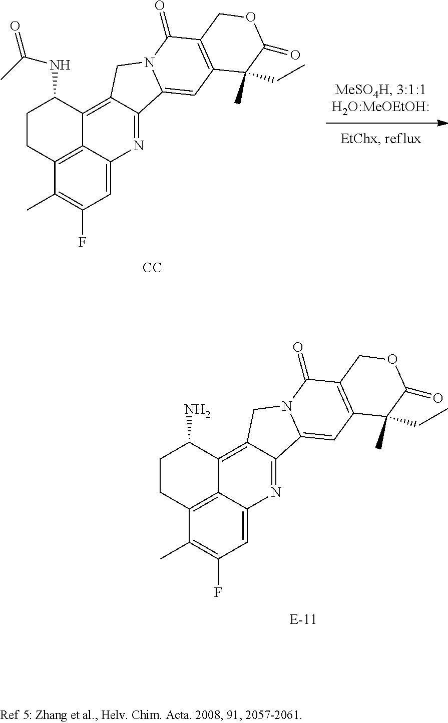

### Proposed Synthesis E-12

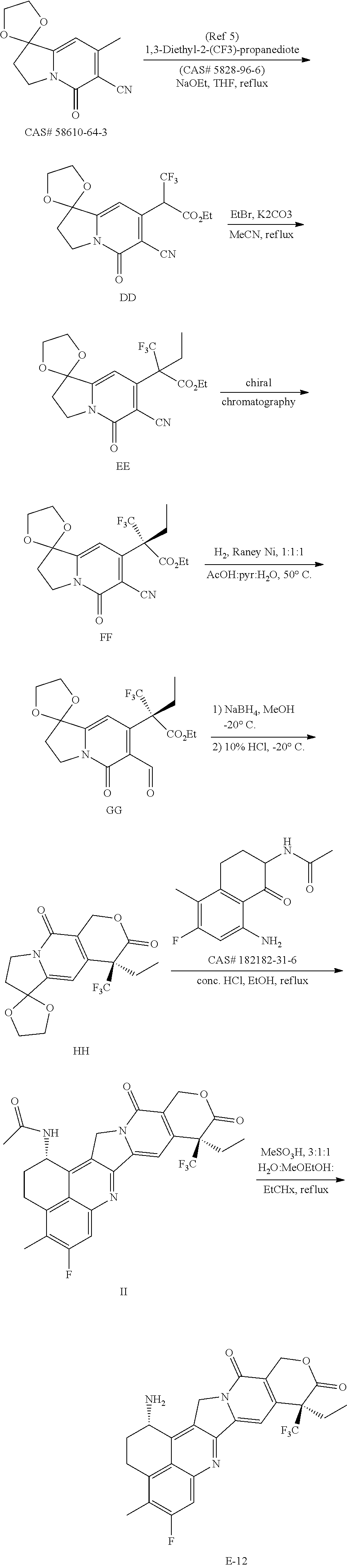

### Proposed Synthesis E-14

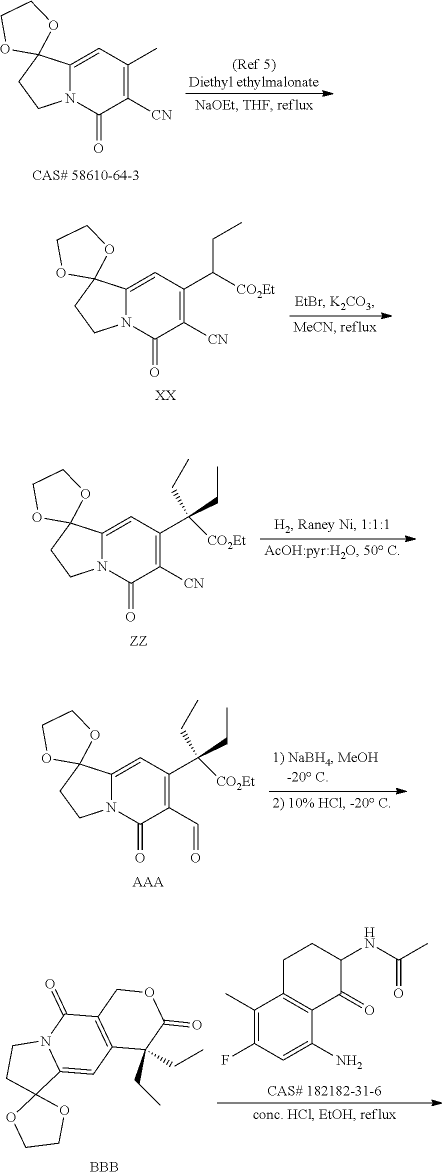

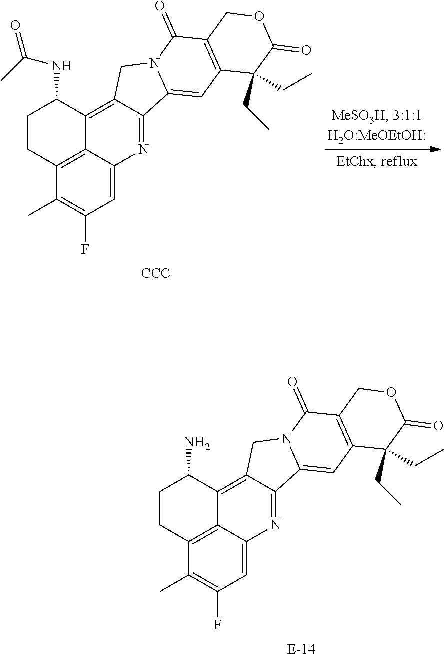

TABLE 1
Exemplary topoisomerase inhibitor compounds of the compound of
Formula (1) and Formula (1′):

| | |
|---|---|
| E-1 | 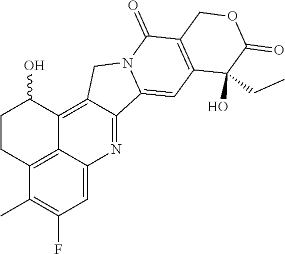 [SMILES: CC[C@@]1(O)C(=O)OCc2c1cc1n(c2=O)Cc2c-1nc1cc(F)c(C)c3c1c2C(O)CC3] |
| E-1a | 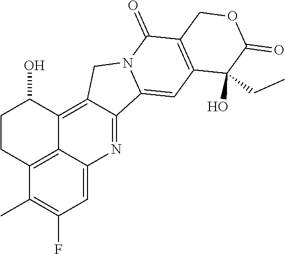 [SMILES: CC[C@@]1(O)C(=O)OCc2c1cc1n(c2=O)Cc2c-1nc1cc(F)c(C)c3c1c2[C@@H](O)CC3] |
| E-1b | 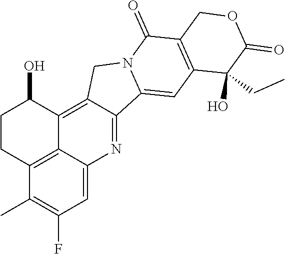 [SMILES: CC[C@@]1(O)C(=O)OCc2c1cc1n(c2=O)Cc2c-1nc1cc(F)c(C)c3c1c2[C@H](O)CC3] |
| E2 | 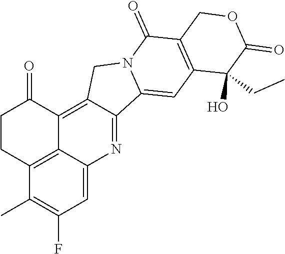 [SMILES: CC[C@@]1(O)C(=O)OCc2c1cc1n(c2=O)Cc2c-1nc1cc(F)c(C)c3c1c2C(=O)CC3] |
| E-3 | 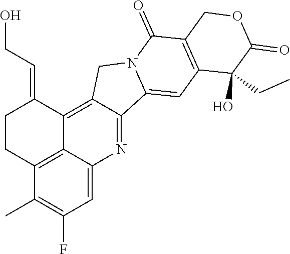 [SMILES: CC[C@@]1(O)C(=O)OCc2c1cc1n(c2=O)Cc2c-1nc1cc(F)c(C)c3c1c2/C(=C/CO)CC3] |
| E-4 | 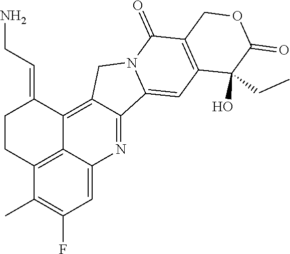 [SMILES: CC[C@@]1(O)C(=O)OCc2c1cc1n(c2=O)Cc2c-1nc1cc(F)c(C)c3c1c2/C(=C/CN)CC3] |
| E-5 | 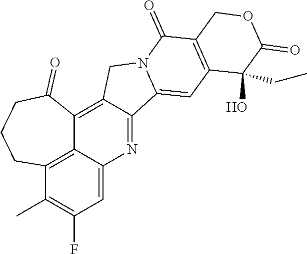 [SMILES: CC[C@@]1(O)C(=O)OCc2c1cc1n(c2=O)Cc2c-1nc1cc(F)c(C)c3c1c2C(=O)CCC3] |
| E-6 | 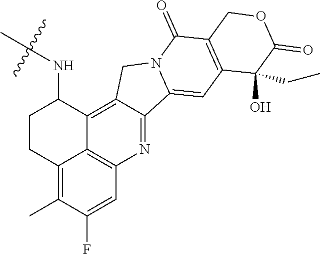 [SMILES: *NC1CCc2c(C)c(F)cc3nc4c(c1c23)Cn1c-4cc2c(c1=O)COC(=O)[C@]2(O)CC] |
| E-7 | 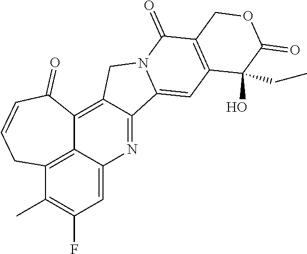 [SMILES: CC[C@@]1(O)C(=O)OCc2c1cc1n(c2=O)Cc2c-1nc1cc(F)c(C)c3c1c2C(=O)C=CC3] |
| E-8 | 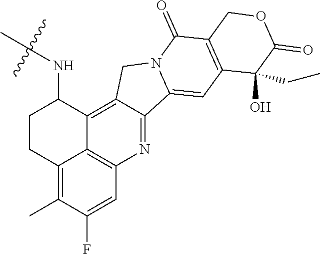 [SMILES: *NC1CCc2c(C)c(F)cc3nc4c(c1c23)Cn1c-4cc2c(c1=O)COC(=O)[C@]2(O)CC] |
| E-9 | 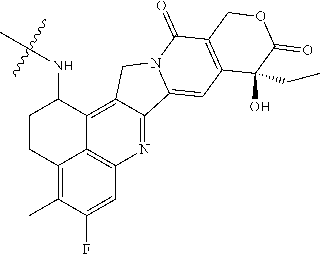 [SMILES: *NC1CCc2c(C)c(F)cc3nc4c(c1c23)Cn1c-4cc2c(c1=O)COC(=O)[C@]2(O)CC] |
| E-10 | 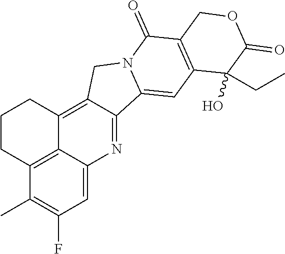 [SMILES: CCC1(O)C(=O)OCc2c1cc1n(c2=O)Cc2c-1nc1cc(F)c(C)c3c1c2CCC3] |
| E-11 | 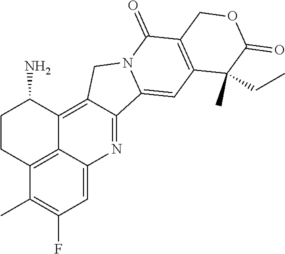 [SMILES: CC[C@@]1(C)C(=O)OCc2c1cc1n(c2=O)Cc2c-1nc1cc(F)c(C)c3c1c2[C@@H](N)CC3] |
| E-12 | 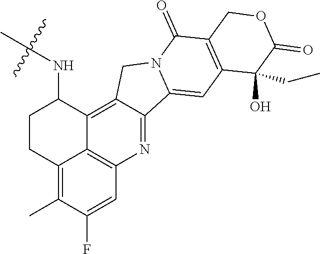 [SMILES: *NC1CCc2c(C)c(F)cc3nc4c(c1c23)Cn1c-4cc2c(c1=O)COC(=O)[C@]2(O)CC] |
| E-14 | 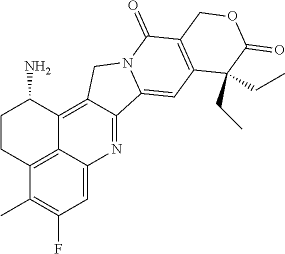 |
| E-15 | 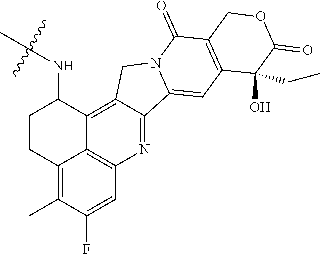 [SMILES: *NC1CCc2c(C)c(F)cc3nc4c(c1c23)Cn1c-4cc2c(c1=O)COC(=O)[C@]2(O)CC] |
| E-17 | 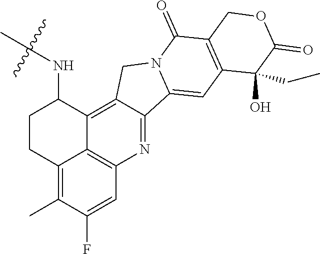 [SMILES: *NC1CCc2c(C)c(F)cc3nc4c(c1c23)Cn1c-4cc2c(c1=O)COC(=O)[C@]2(O)CC] |
| E-18 | 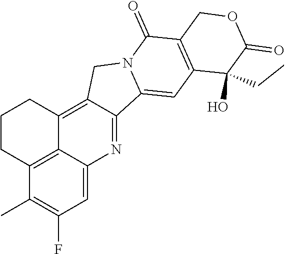 [SMILES: CC[C@@]1(O)C(=O)OCc2c1cc1n(c2=O)Cc2c-1nc1cc(F)c(C)c3c1c2CCC3] |
| E-19 | 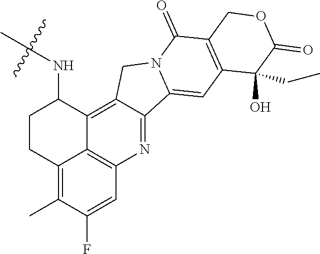 [SMILES: *NC1CCc2c(C)c(F)cc3nc4c(c1c23)Cn1c-4cc2c(c1=O)COC(=O)[C@]2(O)CC] |
| E-20 | 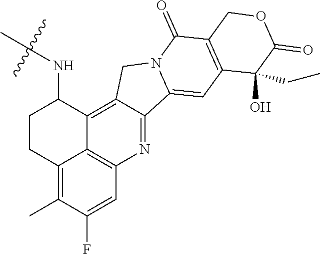 [SMILES: *NC1CCc2c(C)c(F)cc3nc4c(c1c23)Cn1c-4cc2c(c1=O)COC(=O)[C@]2(O)CC] |
| E-21 | 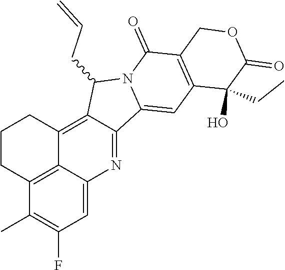 [SMILES: C=CCC1c2c(nc3cc(F)c(C)c4c3c2CCC4)-c2cc3c(c(=O)n21)COC(=O)[C@]3(O)CC] |
| E-22 | 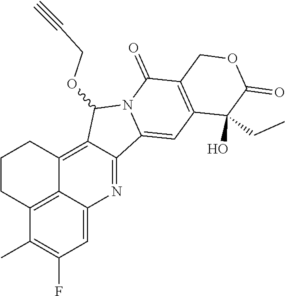 [SMILES: C#CCOC1c2c(nc3cc(F)c(C)c4c3c2CCC4)-c2cc3c(c(=O)n21)COC(=O)[C@]3(O)CC] |
| E-23 | 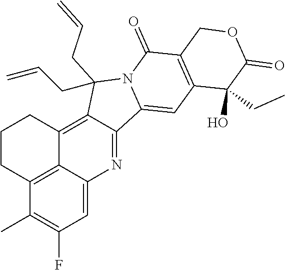 [SMILES: C=CCC1(CC=C)c2c(nc3cc(F)c(C)c4c3c2CCC4)-c2cc3c(c(=O)n21)COC(=O)[C@]3(O)CC] |
| E-24 | 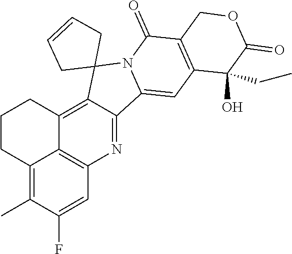 [SMILES: CC[C@@]1(O)C(=O)OCc2c1cc1n(c2=O)C2(CC=CC2)c2c-1nc1cc(F)c(C)c3c1c2CCC3] |
| E-25 | 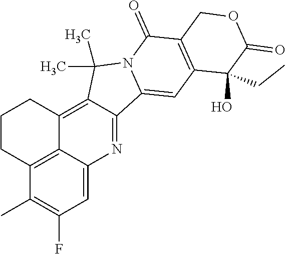 [SMILES: CC[C@@]1(O)C(=O)OCc2c1cc1n(c2=O)C(C)(C)c2c-1nc1cc(F)c(C)c3c1c2CCC3] |
| E-26 | 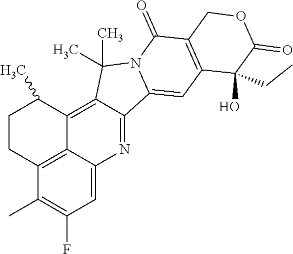 [SMILES: CC[C@@]1(O)C(=O)OCc2c1cc1n(c2=O)C(C)(C)c2c-1nc1cc(F)c(C)c3c1c2C(C)CC3] |
| E-27 | 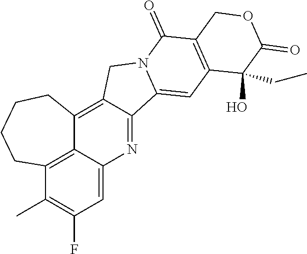 [SMILES: CC[C@@]1(O)C(=O)OCc2c1cc1n(c2=O)Cc2c-1nc1cc(F)c(C)c3c1c2CCCC3] |
| E-28 | 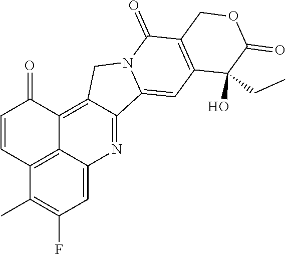 [SMILES: CC[C@@]1(O)C(=O)OCc2c1cc1n(c2=O)Cc2c-1nc1cc(F)c(C)c3c1c2C(=O)C=C3] |
| E-29 | 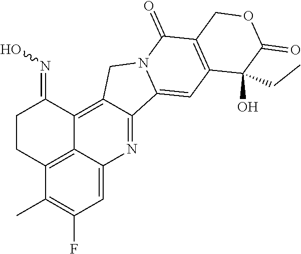 [SMILES: CC[C@@]1(O)C(=O)OCc2c1cc1n(c2=O)Cc2c-1nc1cc(F)c(C)c3c1c2C(=NO)CC3] |
| E-30 | 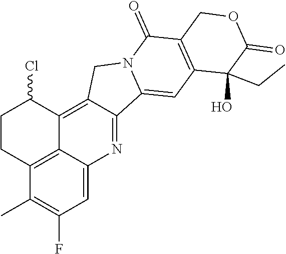 [SMILES: CC[C@@]1(O)C(=O)OCc2c1cc1n(c2=O)Cc2c-1nc1cc(F)c(C)c3c1c2C(Cl)CC3] |
| E-31 | 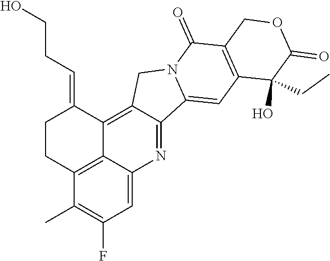 [SMILES: CC[C@@]1(O)C(=O)OCc2c1cc1n(c2=O)Cc2c-1nc1cc(F)c(C)c3c1c2/C(=C/CCO)CC3] |
| E-32 | 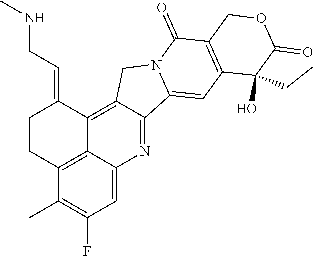 [SMILES: CC[C@@]1(O)C(=O)OCc2c1cc1n(c2=O)Cc2c-1nc1cc(F)c(C)c3c1c2/C(=C/CNC)CC3] |
| E-33 | 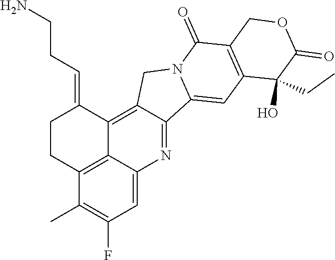 [SMILES: CC[C@@]1(O)C(=O)OCc2c1cc1n(c2=O)Cc2c-1nc1cc(F)c(C)c3c1c2/C(=C/CCN)CC3] |
| E-34 | 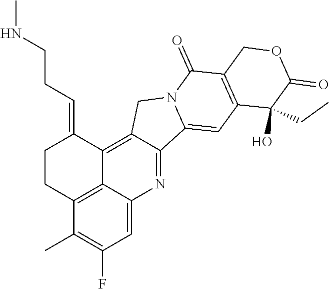 [SMILES: CC[C@@]1(O)C(=O)OCc2c1cc1n(c2=O)Cc2c-1nc1cc(F)c(C)c3c1c2/C(=C/CCNC)CC3] |
| E-38 | 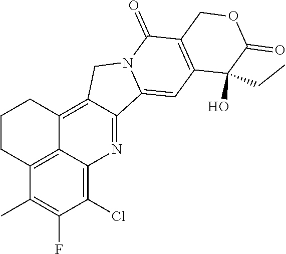 [SMILES: CC[C@@]1(O)C(=O)OCc2c1cc1n(c2=O)Cc2c-1nc1c(Cl)c(F)c(C)c3c1c2CCC3] |

TABLE 2
Exemplary topoisomerase inhibitors of the compound of Formula (A) comprising-L-RX groups.

| | |
|---|---|
| E-35 | 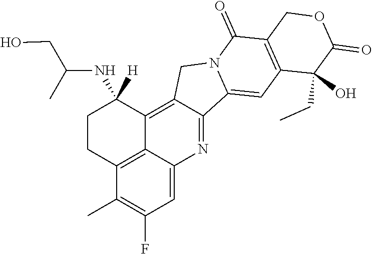 [SMILES: CC[C@@]1(O)C(=O)OCc2c1cc1n(c2=O)Cc2c-1nc1cc(F)c(C)c3c1c2[C@@H](NC(C)CO)CC3] |
| E-35a* | 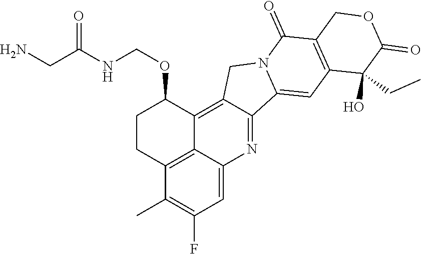 [SMILES: CC[C@@]1(O)C(=O)OCc2c1cc1n(c2=O)Cc2c-1nc1cc(F)c(C)c3c1c2[C@H](OCNC(=O)CN)CC3] |
| E-35b* | 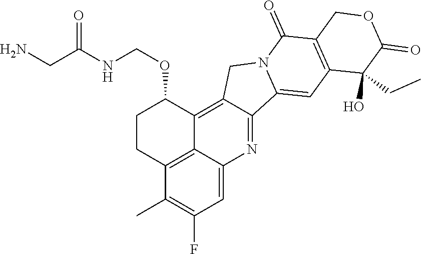 [SMILES: CC[C@@]1(O)C(=O)OCc2c1cc1n(c2=O)Cc2c-1nc1cc(F)c(C)c3c1c2[C@@H](OCNC(=O)CN)CC3] |
| E-36 | 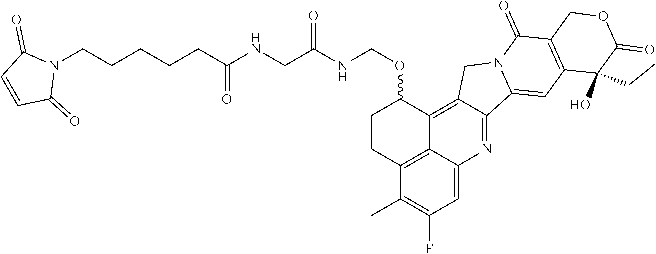 [SMILES: CC[C@@]1(O)C(=O)OCc2c1cc1n(c2=O)Cc2c-1nc1cc(F)c(C)c3c1c2C(OCNC(=O)CNC(=O)CCCCCN1C(=O)C=CC1=O)CC3] |
| E-37 | 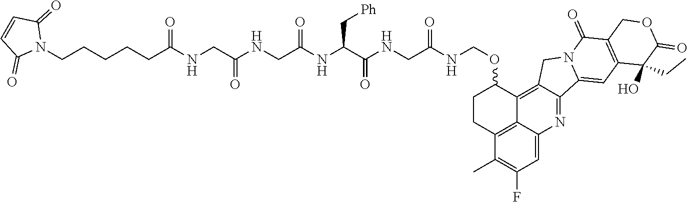 [SMILES: CC[C@@]1(O)C(=O)OCc2c1cc1n(c2=O)Cc2c-1nc1cc(F)c(C)c3c1c2C(OCNC(=O)CNC(=O)[C@H](Cc1ccccc1)NC(=O)CNC(=O)CNC(=O)CCCCCN1C(=O)C=CC1=O)CC3] |
| E-39* | 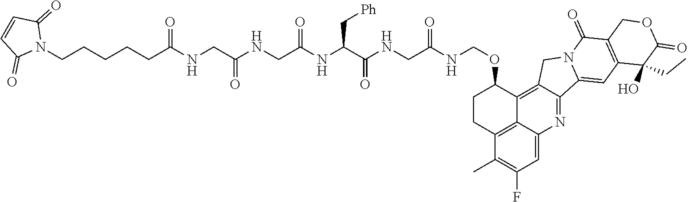 [SMILES: CC[C@@]1(O)C(=O)OCc2c1cc1n(c2=O)Cc2c-1nc1cc(F)c(C)c3c1c2[C@H](OCNC(=O)CNC(=O)[C@H](Cc1ccccc1)NC(=O)CNC(=O)CNC(=O)CCCCCN1C(=O)C=CC1=O)CC3] |
| E-40* | 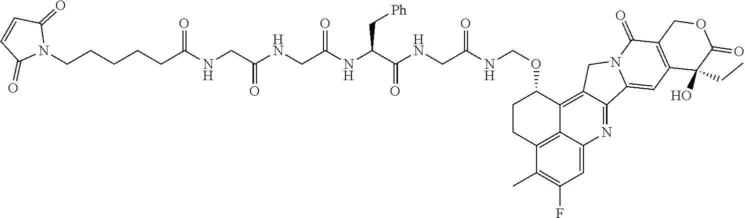 [SMILES: CC[C@@]1(O)C(=O)OCc2c1cc1n(c2=O)Cc2c-1nc1cc(F)c(C)c3c1c2[C@@H](OCNC(=O)CNC(=O)[C@H](Cc1ccccc1)NC(=O)CNC(=O)CNC(=O)CCCCCN1C(=O)C=CC1=O)CC3] |
| E-41 | 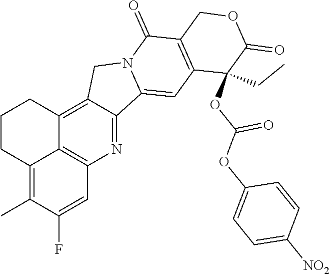 [SMILES: CC[C@@]1(OC(=O)Oc2ccc([N+](=O)[O-])cc2)C(=O)OCc2c1cc1n(c2=O)Cc2c-1nc1cc(F)c(C)c3c1c2CCC3] |
| E-42 | 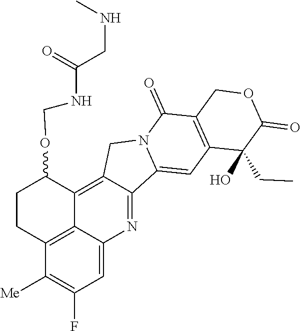 [SMILES: CC[C@@]1(O)C(=O)OCc2c1cc1n(c2=O)Cc2c-1nc1cc(F)c(C)c3c1c2C(OCNC(=O)CNC)CC3] |
| E-43 | 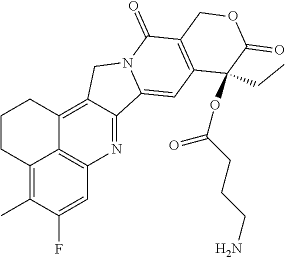 [SMILES: CC[C@@]1(OC(=O)CCCN)C(=O)OCc2c1cc1n(c2=O)Cc2c-1nc1cc(F)c(C)c3c1c2CCC3] |
| E-44 | 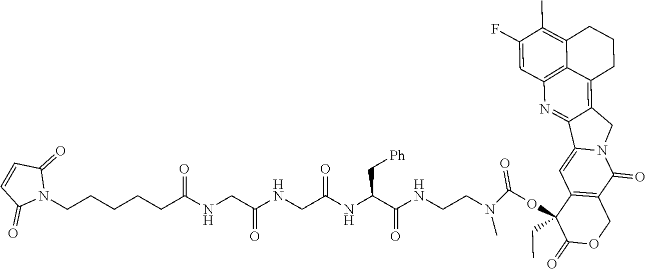 [SMILES: CC[C@@]1(OC(=O)N(C)CCNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3] |
| E-45 | 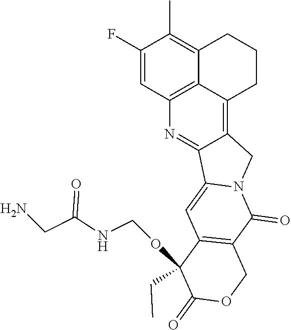 [SMILES: CC[C@@]1(OCNC(=O)CN)C(=O)OCc2c1cc1n(c2=O)Cc2c-1nc1cc(F)c(C)c3c1c2CCC3] |
| E-46 | 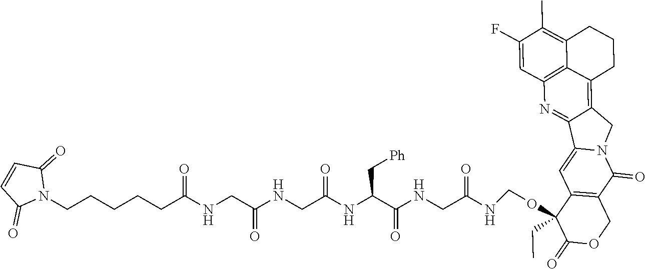 [SMILES: CC[C@@]1(OCNC(=O)CNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3] |
| E-47 | 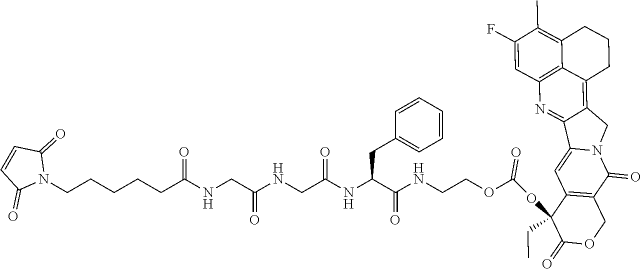 [SMILES: CC[C@@]1(OC(=O)OCCNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3] |
| E-48 | 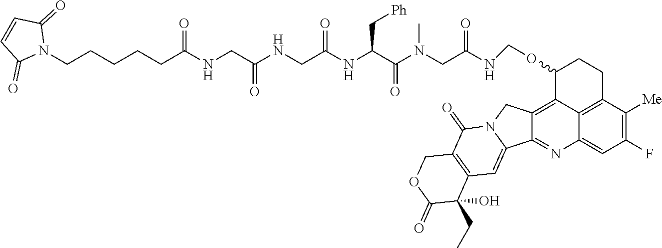 [SMILES: CC[C@@]1(O)C(=O)OCc2c1cc1n(c2=O)Cc2c-1nc1cc(F)c(C)c3c1c2C(OCNC(=O)CN(C)C(=O)[C@H](Cc1ccccc1)NC(=O)CNC(=O)CNC(=O)CCCCCN1C(=O)C=CC1=O)CC3] |
| E-49 | 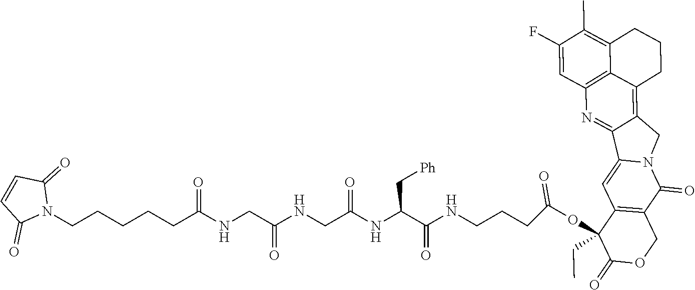 [SMILES: CC[C@@]1(OC(=O)CCCNC(=O)[C@H](Cc2ccccc2)NC(=O)CNC(=O)CNC(=O)CCCCCN2C(=O)C=CC2=O)C(=O)OCc2c1cc1n(c2=O)Cc2c-1nc1cc(F)c(C)c3c1c2CCC3] |

### Example 2: In Vitro E-35 Plasma Stability Study

The decomposition rate of E-35 to E-1 at the concentration of 0.1 mM at 37° C. was evaluated in 1) pH=6.0 1×PBS Buffer. 2) pH=8.0 1×PBS Buffer. 3) pH=7.4 1×PBS Buffer, IgG-depleted rat plasma up to 7 days by ESI LCMS. (3.3% v/v DMSO for all conditions).

No evidence of decomposition was observed over the 7 days.

### Example 3: 2D-Cytotoxicity Assay

Protocol: Day 1:5,000 cells/well were plated and incubated at 37° C. overnight, unless otherwise noted.

Day 2: Payload/vehicle control dilutions were made in respective media and added to cells. Volume added: 50 μL to each well. Starting treatment concentration was 0.1 mM for free payloads/10% for vehicle control and then diluted 10-fold down for a total of 11 treatment dilutions. Each concentration was analyzed in triplicates. Media-only wells was used as a control to calculate percent viability.

Day 5: Cell titer glow reagent (volume: 50 μL) was added to each well, the plate was shaken for 5 mins and the luminescence was recorded.

Drug Treatment Time: 72 hours.

Data Analysis: Percent viability was calculated by dividing the luminescence signal obtained for each treated well by the untreated well (media-only control) and multiplying by 100. Data was next transformed using X=Log (x) and then analyzed with nonlinear regression (curve fit), Dose Response inhibition−log (inhibitor) vs response (3 parameters) using PRISM software to determine the IC₅₀ value. The results of cytotoxicity of SKBR-3 cell line (human breast cancer) are provided in Table 3.

TABLE 3

| Order of | | IC50 |
|---|---|---|
| Potency | Payload | (nM) |
| 1 | E-18 | 1.097 |
| 2 | Exatecan Mesylate | 2.449 |
| 3 | E-10 | 2.701 |
| 4 | E-1 | 3.275 |
| 5 | Exatecan Glycolamide | 5.062 |
| 6 | E-35 | 7.68 |
| 7 | E-42 (1000 cells/well) | 11.96 |
| 8 | E-29 | 15.54 |
| 9 | E-2 (ACN:Water) | 27.73 |
| 10 | E-35a* (2000 cells/well) | 28.1 |
| 11 | E-43 (2000 cells/well) | 32.48 |
| 12 | E-38 | 35.05 |
| 13 | E-17 | 39.92 |
| 14 | E-35b* (2000 cells/well) | 58.75 |
| 15 | E-2 (DMSO) | 73.21 |
| 16 | E-19 | 75.74 |
| 17 | E-22 | 76.26 |
| 18 | E-20 | 83.31 |
| 19 | E-15 | 187 |
| 20 | E-21 | 415 |
| 21 | E-24 | 517 |
| 22 | E-25 | 3035 |
| 23 | E-23 | 3576 |

### ADC Synthesis General Methods

Drug to Antibody Ratio (DAR) by RP-LC/MS was determined by analytical liquid chromatography-mass spectrometry (LC-MS), employing a Waters Acquity Ultra Performance LC system and a Synapt high-definition mass spectrometer. 15 μg of ADC was injected over a PLRP-S column (1000 Å, 8 μm, 2.1×50 mm) against a 25-45% gradient (ACN+0.1% formic acid; 0.35 mL/min flow). Monomeric purity was found via size exclusion chromatography, employed on an Agilent 1260 HPLC. 20 μg of ADC was injected over a TSKgelG3000SWXL column against a 100% PBS gradient (0.8 mL/min flow). The final solution was filtered using 0.2 μm syringe filter. Final analysis was performed on an aliquoted sample of the filtered ADC. Bioconjugates were reduced with dithiothritol (0.5 M; 1 L/10 μg ADC) and allowed to incubate at 37° C.×30 min. Reduced light chain and heavy chain charge envelopes were deconvoluted and reduced light chain and heavy chain species were identified and input into the formula: DAR=[2×(L0+L1)]+[2×(H0+ . . . +H3)].

### Synthesis of HER-E-36

A stock solution of Herceptin (HER, CAS #180288-69-1; 2.5 mg, 0.56 mL, 4.5 mg/mL) was buffer exchanged into 1×PBS, 10 mM EDTA (pH 7.4) with desalting filtration columns (2 mL; 10K MWCO). Tris(2-carboxyethyl) phosphine (TCEP) solution (25 mL, 10 mM) was added to the buffer-exchanged antibody solution (2.5 mg, 0.56 mL, 4.5 mg/mL) and allowed to incubate at room temperature for two hours. Following reduction, the solution was buffer exchanged into fresh 1×PBS, 10 mM EDTA (pH 7.4). A solution of E-36 (13.3 mL, 10 mM) was added to the 1.0 mg of reduced antibody solution, with polysorbate 80 spiked into the solution to achieve 1% v/v and allowed to incubate at room temperature for two hours.

### Purification of HER-E-36

The crude ADC solution was dialyzed against PBS buffer using 10-kDa MWCO, PD-10 gravity flow columns to remove free payload, buffer additives, and TCEP. The calculated DAR of purified HER-E-36 was 8.0.

### Synthesis of HER-E-37

A stock solution of Herceptin (HER, CAS #180288-69-1; 2.5 mg, 0.56 mL, 4.5 mg/mL) was buffer exchanged into 1×PBS, 10 mM EDTA (pH 7.4) with desalting filtration columns (2 mL; 10K MWCO). Tris(2-carboxyethyl) phosphine (TCEP) solution (25 mL, 10 mM) was added to the buffer-exchanged antibody solution (2.5 mg, 0.56 mL, 4.5 mg/mL) and allowed to incubate at room temperature for two hours. Following reduction, the solution was buffer exchanged into fresh 1×PBS, 10 mM EDTA (pH 7.4). A solution of E-37 (13.3 mL, 10 mM) was added to the 1.0 mg of reduced antibody solution, with polysorbate 80 spiked into the solution to achieve 1% v/v and allowed to incubate at room temperature for two hours.

### Purification of HER-E-37

The crude ADC solution was dialyzed against PBS buffer using 10-kDa MWCO, PD-10 gravity flow columns to remove free payload, buffer additives, and TCEP. The calculated DAR of purified HER-E-37 was 8.0.

### Synthesis of HER-E-39

A stock solution of Herceptin (HER, CAS #180288-69-1; 3 mg, 0.67 mL, 4.5 mg/mL) was prepared in 1×PBS, (pH 7.4). Tris(2-carboxyethyl) phosphine (TCEP) solution (20 μL, 10 mM, 10 eq) was added to the antibody solution (3 mg, 0.67 mL, 4.5 mg/mL) with 10% DTPA (67 μl, 10 mM) and allowed to reduce at 37° C. for 90 min. A solution of E-39 (40 μL-20 eq, 10 mM) was added to the 3.0 mg of reduced antibody solution along with 10% DMA (67 μl) allowed to incubate at room temperature for two hours on rotor for end-to-end mixing. Crude analysis at 2 hours for DAR using RP-LC/MS showed DAR 7.7.

### Purification of HER-E-39

The HER-E-39 crude ADC solution was dialyzed against 1×-PBS buffer, pH-7.4 using 0.5 ml dialysis cassette generation 2 at room temperature for 5-6 h followed up overnight at cold room with continuous stirring using magnetic stirrer to remove free payload, buffer additives, and TCEP. The calculated DAR of purified HER-E-39 ADC was 7.7.

### Synthesis of HER-E-40

A stock solution of Herceptin (HER, CAS #180288-69-1; 3 mg, 0.67 mL, 4.5 mg/mL) was prepared in 1×PBS, (pH 7.4). Tris(2-carboxyethyl) phosphine (TCEP) solution (20 μL, 10 mM, 10 eq) was added to the antibody solution (3 mg, 0.67 mL, 4.5 mg/mL) with 10% DTPA (67 μl, 10 mM) and allowed to reduce at 37° C. for 90 min. A solution of E-40 (40 μL-20 eq, 10 mM) was added to the 3.0 mg of reduced antibody solution along with 10% DMA (67 μl) allowed to incubate at room temperature for two hours on rotor for end-to-end mixing. Crude analysis at 2 hours for DAR using RP-LC/MS showed DAR 7.8.

### Purification of HER-E-40

The HER-E-40 crude ADC solution was dialyzed against 1×-PBS buffer, pH-7.4 using 0.5 ml dialysis cassette generation 2 at room temperature for 5-6 h followed up overnight at cold room with continuous stirring using magnetic stirrer to remove free payload, buffer additives, and TCEP. The calculated DAR of purified HER-E-40 ADC was 7.8.

### Synthesis of HER-E-44

A stock solution of Herceptin (HER, CAS #180288-69-1; 1 mg, 0.23 mL, 4.4 mg/mL) was prepared in 1×PBS, (pH 7.4). Tris(2-carboxyethyl) phosphine (TCEP) solution (6.7 μL, 10 mM, 10 eq) was added to the antibody solution (1 mg, 0.23 mL, 4.4 mg/mL) and allowed to reduce at 37° C. for 90 min. A solution of E-44 (6.7 μL-10 eq, 10 mM) was added to the 1.0 mg of reduced antibody solution along with 20% DMA (52 μl) allowed to incubate at room temperature for two hours on rotor for end-to-end mixing. Crude analysis at 2 hours for DAR using RP-LC/MS showed DAR 8.0.

### Purification of HER-E-44

The HER-E-44 crude ADC solution was dialyzed against 1×-PBS buffer, pH-7.4 using 0.5 ml dialysis cassette generation 2 at room temperature for 5-6 h followed up overnight at cold room with continuous stirring using magnetic stirrer to remove free payload, buffer additives, and TCEP. The calculated DAR of purified HER-E-44 was 7.9.

### Synthesis of HER-E-46

A stock solution of Herceptin (HER, CAS #180288-69-1; 1 mg, 0.23 mL, 4.4 mg/mL) was prepared in 1×PBS, (pH 7.4). Tris(2-carboxyethyl) phosphine (TCEP) solution (6.7 μL, 10 mM, 10 eq) was added to the antibody solution (1 mg, 0.23 mL, 4.4 mg/mL) and allowed to reduce at 37° C. for 90 min. A solution of E-46 (13.34 μL-20 eq, 10 mM) was added to the 1.0 mg of reduced antibody solution along with 10% DMA (25 μl) allowed to incubate at room temperature for two hours on rotor for end-to-end mixing. Crude analysis at 2 hours for DAR using RP-LC/MS showed DAR 8.3.

### Purification of HER-E-46

The HER-E-46 crude ADC solution was dialyzed against 1×-PBS buffer, pH-7.4 using 0.5 ml dialysis cassette generation 2 at room temperature for 5-6 h followed up overnight at cold room with continuous stirring using magnetic stirrer to remove free payload, buffer additives, and TCEP. The calculated DAR of purified HER-E-46 was 8.3.

### Synthesis of HER-E-48

A stock solution of Herceptin (HER, CAS #180288-69-1; 1 mg, 0.23 mL, 4.4 mg/mL) was prepared in 1×PBS, (pH 7.4). Tris(2-carboxyethyl) phosphine (TCEP) solution (6.7 μL, 10 mM, 10 eq) was added to the antibody solution (1 mg, 0.23 mL, 4.4 mg/mL) and allowed to reduce at 37° C. for 90 min. A solution of E-48 (13.34 μL-20 eq, 10 mM) was added to the 1.0 mg of reduced antibody solution along with 20% DMA (52 μl) allowed to incubate at room temperature for two hours on rotor for end-to-end mixing. Crude analysis at 2 hours for DAR using RP-LC/MS showed DAR 7.9.

### Purification of HER-E-48

The crude HER-E-48 solution was dialyzed against 1×-PBS buffer, pH-7.4 using 0.5 ml dialysis cassette generation 2 at room temperature for 5-6 h followed up overnight at cold room with continuous stirring using magnetic stirrer to remove free payload, buffer additives, and TCEP. The calculated DAR of purified HER-E-48 was 7.9.

### Synthesis of HER-E-49

A stock solution of Herceptin (HER, CAS #180288-69-1; 1 mg, 0.23 mL, 4.4 mg/mL) was prepared in 1×PBS, (pH 7.4). Tris(2-carboxyethyl) phosphine (TCEP) solution (6.7 μL, 10 mM, 10 eq) was added to the antibody solution (1 mg, 0.23 mL, 4.4 mg/mL) and allowed to reduce at 37° C. for 90 min. A solution of E-49 (16.75 μL-25 eq, 10 mM) was added to the 1.0 mg of reduced antibody solution along with 20% DMA and 20% Propylene glycol (50 μl) allowed to incubate at room temperature for two hours on rotor for end-to-end mixing. Crude analysis at 2 hours for DAR using RP-LC/MS showed DAR 6.7 with 81% monomer purity.

### Purification of HER-E-49

The crude HER-E-49 ADC further purified using Size exclusion chromatography to remove aggregate, free payload, buffer additives, and TCEP. The final solution was concentrated down using 30 KDa concentrator at 5500 g for 5 min and then filtered using 0.2 μm syringe filter. The calculated DAR of purified HER-E-49 was 2.7.

TABLE 4
IC50 Values (pM)

| Cell | HER-E-36 | HER-E-37 | HER-E-39 | HER-E-40 | HER-E-44 |
|---|---|---|---|---|---|
| Line | (DAR 8) | (DAR 8) | (DAR 7.7) | (DAR 7.8) | (DAR 7.9) |
| SKBR-3 | 790.08 pM | 47.82 pM | 44.88 pM | 36.15 pM | Not Tested |

| | | | | HER- | HER- |
|---|---|---|---|---|---|
| Cell | HER-E-46 | HER-E-48 | HER-E-49 | Vedotin | Deruxtecan |
| Line | (DAR 8.3) | (DAR 7.9) | (DAR 2.7) | (DAR 4.5) | (DAR 8) |
| SKBR-3 | 35.06 pM | 109.2 pM | Not Tested | 31.18 pM | 59.46 pM |

| Cell | |
|---|---|
| Line | Herceptin |
| SKBR-3 | N.A. |

### SEQUENCE LISTING

### SEQ ID NO: 1

GGFG
Organism: artificial
Comment: amino acid spacer

The disclosed aspects herein include at least the following:

Aspect 1: A compound of Formula (1), Formula (1′), or a pharmaceutically acceptable salt thereof,

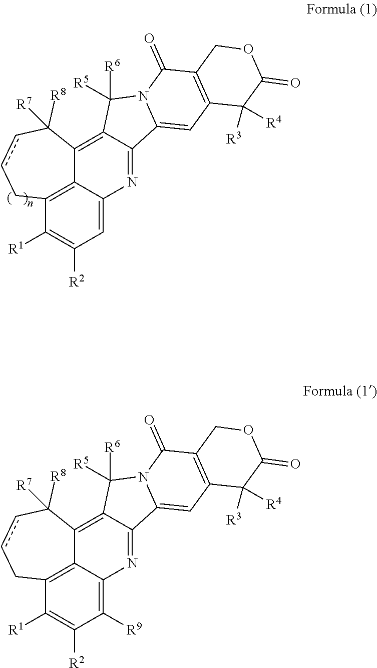

wherein
   R¹ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R¹ is C₁₋₆ alkyl, and more specifically R¹ is C₁ alkyl;
   R² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R² is halogen, and more specifically R² is F;
   R³ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁-6 haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R³ is hydroxyl, C₁₋₃ alkyl, or C₁ haloalkyl, and more specifically R³ is hydroxyl;
   R⁴ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y′ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic, specifically R⁴ is C₁₋₃ alkyl or C₁₋₃ haloalkyl, and more specifically R⁴ is C₂ alkyl;
   wherein at least one of R³ and R⁴ is not H;
   R⁵ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁-6 haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁵ is H, hydroxyl, C₁₋₃ alkyl, C₂₋₃ alkenyl, C₂₋₃ alkynyl, (C₂₋₃ alkenyl)O—, or (C₂₋₃ alkynyl)O—, more specifically R⁵ is H, hydroxyl, or C₁ alkyl;
   R⁶ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁶ is H, hydroxyl, C₁₋₃ alkyl, C₂₋₃ alkenyl, C₂₋₃ alkynyl, —O(C₂₋₃ alkenyl), or —O(C₂₋₃ alkynyl), more specifically R⁶ is H, hydroxyl, or C₁ alkyl;
   or R⁵ and R⁶ together form an optionally substituted 5-6 member cyclic structure that is saturated or unsaturated, specifically a C₅₋₆ cycloalkyl or a C₅₋₆ cycloalkenyl;
   R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, —O—(C₁₋₃ alkyl)-NH—(C═O)—(C₁₋₃ alkyl)-NH₂, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O)—, specifically R⁷ is H, hydroxyl, amino, C₁₋₃ alkyl, or halogen, more specifically R⁷ is H, hydroxyl, amino, C₁ alkyl, or chloro;
   R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, —O—(C₁₋₃ alkyl)-NH—(C═O)—(C₁₋₃ alkyl)-NH₂, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—, specifically R⁸ is H, hydroxyl, amino, C₁₋₃ alkyl, or halogen, more specifically R⁸ is H, hydroxyl, amino, C₁ alkyl, or chloro;
   or R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
   R⁹ is H, C₁₋₆ alkyl, or halogen, specifically R⁹ is C₁;
   [symbol] is a single or double bond; and
   n is 0 or 1;
   with the following proviso:
   when R¹ is methyl, R² is fluoro, one of R³ and R⁴ is hydroxyl and the other is ethyl, R⁵ is H, R⁶ is H, [symbol] is a single bond, and n is 0, then when one of R⁷ and R⁸ is H, the other is not H, amino, mono-C₁₋₆ alkylamine, di-C₁₋₆ alkylamine, hydroxyl, or HO—(C₁₋₃ alkyl)-O—.

Aspect 2: The compound of Aspect 1, wherein R¹ is C₁₋₆ alkyl and specifically R¹ is C₁ alkyl.

Aspect 3: The compound of any one of Aspects 1-2, wherein R² is halogen and more specifically R² is F.

Aspect 4: The compound of any one of Aspects 1-3, wherein R¹ is C₁ alkyl and R² is F.

Aspect 5: The compound of any one of Aspects 1-4, wherein R³ is hydroxyl, C₁₋₃ alkyl, or C₁ haloalkyl, and more specifically R³ is hydroxyl.

Aspect 6: The compound of any one of Aspects 1-5, wherein R⁴ is C₁₋₃ alkyl or C₁₋₃ haloalkyl, and more specifically R⁴ is C₂ alkyl.

Aspect 7: The compound of any one of Aspects 1-6, wherein R³ is hydroxyl and R⁴ is C₂ alkyl.

Aspect 8: The compound of any one of Aspects 1-7, wherein R⁵ is H, hydroxyl, C₁₋₃ alkyl, C₂₋₃ alkenyl, C₂₋₃ alkynyl, (C₂₋₃ alkenyl)O—, or (C₂₋₃ alkynyl)O—, more specifically R⁵ is H, hydroxyl, or C₁ alkyl.

Aspect 9: The compound of any one of Aspects 1-8, wherein R⁶ is H, hydroxyl, C₁₋₃ alkyl, C₂₋₃ alkenyl, C₂₋₃ alkynyl, —O(C₂₋₃ alkenyl), or —O(C₂₋₃ alkynyl), more specifically R⁶ is H, hydroxyl, or C₁ alkyl.

Aspect 10: The compound of any one of Aspects 1-7, wherein R⁵ and R⁶ together form an optionally substituted 5-6 member cyclic structure that is saturated or unsaturated, specifically a C₅₋₆ cycloalkyl or a C₅₋₆ cycloalkenyl.

Aspect 11: The compound of any one of Aspects 1-10, wherein R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—.

Aspect 12: The compound of any one of Aspects 1-10, wherein R⁷ is H, hydroxyl, amino, C₁₋₃ alkyl, or halogen, and more specifically R⁷ is H, hydroxyl, C₁ alkyl, or chloro.

Aspect 13: The compound of any one of Aspects 1-12, wherein R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—.

Aspect 14: The compound of any one of Aspects 1-12, wherein R⁸ is H, hydroxyl, amino, C₁₋₃ alkyl, or halogen, more specifically R⁸ is H, hydroxyl, C₁ alkyl, or chloro.

Aspect 15: The compound of any one of Aspects 1-10, wherein R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic.

Aspect 16: The compound of any one of Aspects 1-15, wherein [symbol] is a single bond.

Aspect 17: The compound of any one of Aspects 1-15, wherein [symbol] is a double bond.

Aspect 18: The compound of any one of Aspects 1-17, wherein n is 0.

Aspect 19: The compound of any one of Aspects 1-17, wherein n is 1.

Aspect 20: The compound of any one of Aspects 1-19, wherein neither R⁷ or R⁸ is amino, mono-C₁₋₆ alkylamine, di-C₁₋₆ alkylamine, hydroxyl, or HO—(C₁₋₃ alkyl)-O—.

Aspect 21: A compound of

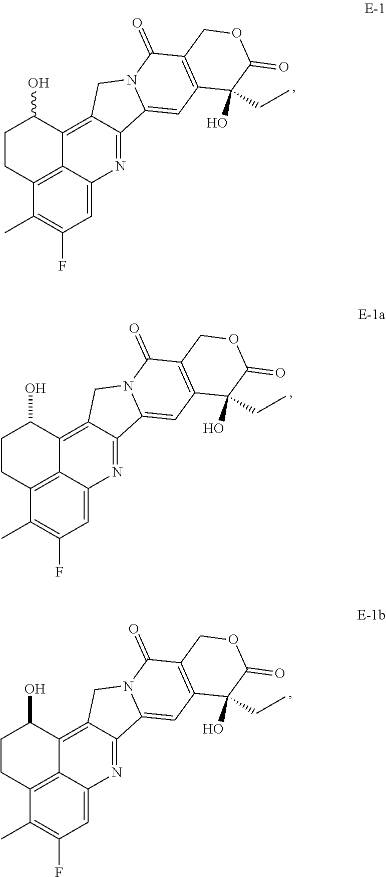 [SMILES: CC[C@@]1(O)C(=O)OCc2c1cc1n(c2=O)Cc2c-1nc1cc(F)c(C)c3c1c2C(O)CC3 ; CC[C@@]1(O)C(=O)OCc2c1cc1n(c2=O)Cc2c-1nc1cc(F)c(C)c3c1c2[C@@H](O)CC3 ; CC[C@@]1(O)C(=O)OCc2c1cc1n(c2=O)Cc2c-1nc1cc(F)c(C)c3c1c2[C@H](O)CC3]

or
a pharmaceutically acceptable salt thereof.

Aspect 22: A compound of Formula (A) or Formula (B)

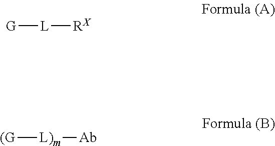

wherein
   L is a linking group;
   G is a structure of Formula (1), Formula (1′) of any one of claims 1-20 or a structure in Table 1, excluding E-1 and E-10, covalently attached to L through one of R¹, R², R³, R⁴, R⁵, R⁶, R⁷, or R⁸, specifically attached through R⁷ or R⁸;
   R^{X} is a reactive group suitable for forming a covalent bond to an antibody or antibody fragment;
   Ab is an antibody or antibody fragment; and
   m is 1, 2, 3, 4, 5, 6, 7, or 8.

Aspect 23: The compound of Aspect 22, wherein L, the linking group, is a bond or a group containing 1 to about 250 non-hydrogen atoms including C, N, O, S, halogen, or a combination thereof; further wherein L can optionally include one or more groups including an ether, thioether, amide, carbonyl, ester, carbonate, carbamate, urea, or a combination thereof. In an embodiment, L comprises an ethylene glycol unit, specifically about 2 to about 25 repeating ethylene glycol units, more specifically about 5 to about 10 repeating ethylene glycol units; an amino acid unit, specifically 1 to about 12 amino acid units, more specifically about 2 to about 10 amino acid units, and yet more specifically about 4 to about 8 amino acid units; or a combination thereof.

Aspect 24: The compound of Aspect 23, wherein the amino acid groups of L, each amino acid unit can be arginine, histidine, lysine, aspartic acid, glutamic acid, serine, threonine, asparagine, glutamine, cysteine, selenocystein, glycine, proline, alanine, valine, isoleucine, leucine, methionine, phenylalanine, tyrosine, tryptophan, or citrulline (Cit).

Aspect 25: The compound of any one of Aspects 22-24, wherein R^{X} is an amine, —O—NH₂, maleimide, azide, 2,5-dioxopyrrolidin-1-yl formate,

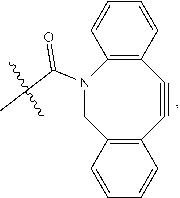 [SMILES: *C(=O)N1Cc2ccccc2C#Cc2ccccc21]

thiol, pentafluorophenyl ester, or a carbonate.

Aspect 26: The compounds of Table 2.

Aspect 27: A pharmaceutical formulation comprising, the compound of any one of Aspects 1-26 and a pharmaceutically acceptable excipient.

Aspect 28: A method of treating a proliferative disease including a cancer, comprising administering to a patient in need thereof the compound of any one of Aspects 1-26 or the pharmaceutical formulation of Aspect 27.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the group(s) includes one or more groups). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A compound of Formula (1), Formula (1′), or a pharmaceutically acceptable salt thereof, 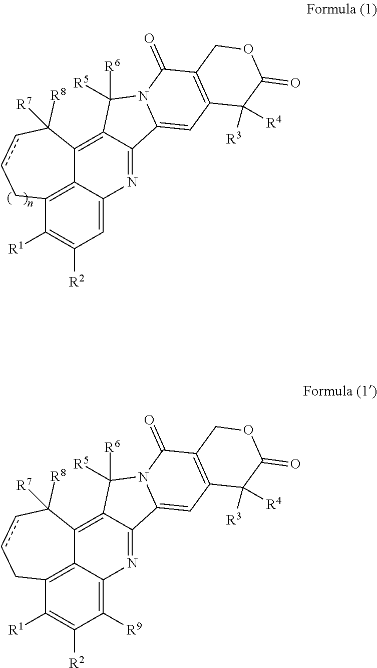
wherein
R¹ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
R² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
R³ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
R⁴ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
wherein at least one of R³ and R⁴ is not H;
R⁵ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—;
R⁶ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—;
or R⁵ and R⁶ together form an optionally substituted 5-6 member cyclic structure that is saturated or unsaturated;
R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, —O—(C₁₋₃ alkyl)-NH—(C═O)—(C₁₋₃ alkyl)-NH₂, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O)—;
R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or —O—(C₁₋₃ alkyl)-NH—(C═O)—(C₁₋₃ alkyl)-NH₂, Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O)—;
or R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic;
R⁹ is H, C₁₋₆ alkyl, or halogen; [symbol]
is a single or double bond; and
n is 0 or 1;
with the following proviso:
when R¹ is methyl, R² is fluoro, one of R³ and R⁴ is hydroxyl and the other is ethyl, R⁵ is H, R⁶ is H, [symbol] is a single bond, and n is 0, then when one of R⁷ and R⁸ is H, the other is not H, amino, mono-C₁₋₆ alkylamine, di-C₁₋₆ alkylamine, hydroxyl, or HO—(C₁₋₃ alkyl)-O—.

2. The compound of claim 1, wherein R¹ is C₁₋₆ alkyl.

3. The compound of claim 1, wherein R² is halogen.

4. The compound of claim 1, wherein R¹ is C₁ alkyl and R² is F.

5. The compound of claim 1, wherein R³ is hydroxyl, C₁₋₃ alkyl, or C₁ haloalkyl.

6. The compound of claim 1, wherein R⁴ is C₁₋₃ alkyl or C₁₋₃ haloalkyl.

7. The compound of claim 1, wherein R³ is hydroxyl and R⁴ is C₂ alkyl.

8. The compound of claim 1, wherein R⁵ is H, hydroxyl, C₁₋₃ alkyl, C₂₋₃ alkenyl, C₂₋₃ alkynyl, (C₂₋₃ alkenyl)O—, or (C₂₋₃ alkynyl)O—.

9. The compound of claim 1, wherein R⁶ is H, hydroxyl, C₁₋₃ alkyl, C₂₋₃ alkenyl, C₂₋₃ alkynyl, —O(C₂₋₃ alkenyl), or —O(C₂₋₃ alkynyl).

10. The compound of claim 1, wherein R⁵ and R⁶ together form an optionally substituted 5-6 member cyclic structure that is saturated or unsaturated.

11. The compound of claim 1, wherein R⁷ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—.

12. The compound of claim 1, wherein R⁷ is H, hydroxyl, amino, C₁₋₃ alkyl, or halogen.

13. The compound of claim 1, wherein R⁸ is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, or Y¹—(C₁₋₆ alkyl)-, wherein Y¹ is C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, (C₂₋₆ alkenyl)O—, or (C₂₋₆ alkynyl)O—.

14. The compound of claim 1, wherein R⁸ is H, hydroxyl, amino, C₁₋₃ alkyl, or halogen.

15. The compound of claim 1, wherein R⁷ and R⁸ together form oxo (O═), HON═, Y²—(C₁₋₆ alkyl)-CH═ wherein Y² is H, C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, C₃-C₈ cycloalkyl, C₄-C₈ cycloalkenyl, aryl, heteroaryl, or C₃-C₇ heterocyclic.

16. The compound of claim 1, wherein [symbol] is a single bond.

17. The compound of claim 1, wherein [symbol] is a double bond.

18. The compound of claim 1, wherein n is 0.

19. The compound of claim 1, wherein n is 1.

20. The compound of claim 1, wherein neither R⁷ or R⁸ is amino, mono-C₁₋₆ alkylamine, di-C₁₋₆ alkylamine, hydroxyl, or HO—(C₁₋₃ alkyl)-O—.

21. A compound of 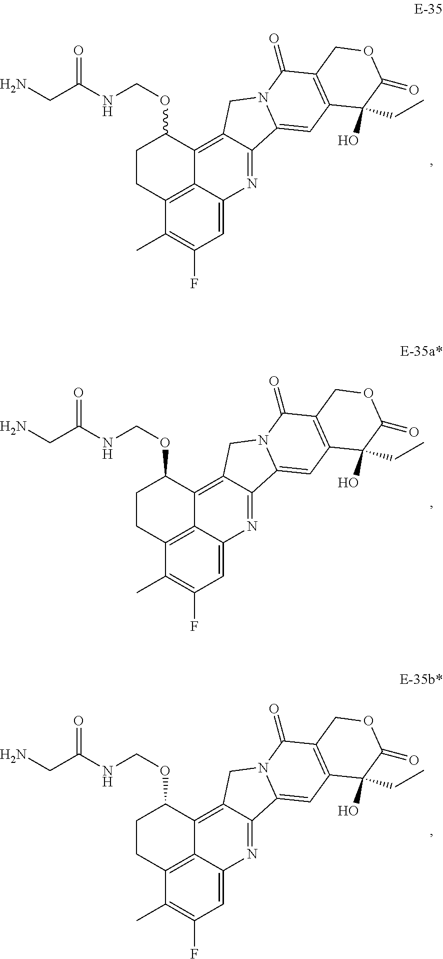 [SMILES: CC[C@@]1(O)C(=O)OCc2c1cc1n(c2=O)Cc2c-1nc1cc(F)c(C)c3c1c2C(OCNC(=O)CN)CC3 ; CC[C@@]1(O)C(=O)OCc2c1cc1n(c2=O)Cc2c-1nc1cc(F)c(C)c3c1c2[C@H](OCNC(=O)CN)CC3 ; CC[C@@]1(O)C(=O)OCc2c1cc1n(c2=O)Cc2c-1nc1cc(F)c(C)c3c1c2[C@@H](OCNC(=O)CN)CC3]
or
a pharmaceutically acceptable salt thereof.

22. A compound of Formula (A) or Formula (B) 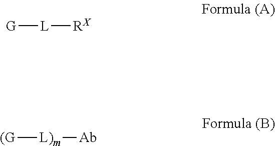
wherein
L is a linking group;
G is a structure of Formula (1) or Formula (1′) of claim 1, a compound of claim 21, or a structure in Table 1, excluding E-1 and E-10, covalently attached to L through one of R¹, R², R³, R⁴, R⁵, R⁶, R⁷, or R⁸;
R^{X} is a reactive group suitable for forming a covalent bond to an antibody or antibody fragment;
Ab is an antibody or antibody fragment; and
m is 1, 2, 3, 4, 5, 6, 7, or 8.

23. The compound of claim 22, wherein L, the linking group, is a bond or a group containing 1 to about 250 non-hydrogen atoms including C, N, O, S, halogen, or a combination thereof; further wherein L can optionally include one or more groups including an ether, thioether, amide, carbonyl, ester, carbonate, carbamate, urea, or a combination thereof. In an embodiment, L comprises an ethylene glycol unit; an amino acid unit; or a combination thereof.

24. The compound of claim 23, wherein the amino acid groups of L, each amino acid unit can be arginine, histidine, lysine, aspartic acid, glutamic acid, serine, threonine, asparagine, glutamine, cysteine, selenocystein, glycine, proline, alanine, valine, isoleucine, leucine, methionine, phenylalanine, tyrosine, tryptophan, or citrulline (Cit).

25. The compound of claim 22, wherein R^{X} is an amine, —O—NH₂, maleimide, azide, 2,5-dioxopyrrolidin-1-yl formate, 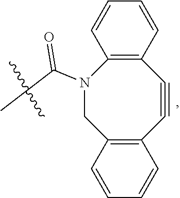 [SMILES: *C(=O)N1Cc2ccccc2C#Cc2ccccc21]
thiol, pentafluorophenyl ester, or a carbonate.

26. A compound as found in Table 2.

27. A pharmaceutical formulation comprising, the compound of claim 1 and a pharmaceutically acceptable excipient.

28. A method of treating a proliferative disease including a cancer, comprising administering to a patient in need thereof the compound of claim 1.
